(19) United States
(12) Patent Application Publication
(10) Pub. No.: US 2018/0267021 A1
(43) Pub. Date: Sep. 20, 2018

(54) METHODS AND DEVICES FOR ASSESSING CELL PROPERTIES UNDER CONTROLLED GAS ENVIRONMENTS

(71) Applicants: Carnegie Mellon University, Pittsburg, PA (US); Massachusetts Institute of Technology, Cambridge, MA (US)

(72) Inventors: Subra SURESH, Pittsburgh, PA (US); E. DU, Boynton Beach, FL (US); Monica DIEZ SILVA, Boston, MA (US); Ming DAO, West Roxbury, MA (US)

(73) Assignees: Carnegie Mellon University, Pittsburg, PA (US); Massachusetts Institute of Technology, Cambridge, MA (US)

(21) Appl. No.: 15/533,277

(22) PCT Filed: Dec. 4, 2015
(86) PCT No.: PCT/US2015/064026

§ 371 Date: Jun. 5, 2017

Related U.S. Application Data
(60) Provisional application No. 62/088,507, filed on Dec. 5, 2014.

(51) Int. Cl. G01N 33/50; G01N 15/10

(52) U.S. Cl. CPC G01N 33/5026; G01N 15/1056; G01N 2800/52; G01N 2015/105; G01N 2015/1006

(57) ABSTRACT

The invention in some aspects relates to high throughput methods and devices for evaluating mechanical, morphological, kinetic, rheological or hematological properties of cells, such as blood cells under regulated gas conditions. In some aspects, the invention relates to methods and devices for diagnosing and/or characterizing a condition or disease in a subject by measuring a property of a cell from the subject, under controlled gas conditions.

### RELATED APPLICATIONS

The present application claims priority under 35 U.S.C. §119(e) to U.S. Provisional Application No. 62/088,507, filed Dec. 5, 2014 which is incorporated herein by reference.

### FEDERALLY SPONSORED RESEARCH

This invention was made with government support under R01 HL094270 and U01 HL114476 awarded by the National Institutes of Health. The government has certain rights in the invention.

### BACKGROUND OF THE INVENTION

A major challenge with in vitro investigations of the pathophysiological processes in sickle cell disease (SCD) has been the lack of a well-controlled microenvironment to mimic in vivo circulating conditions. Sickle cell disease (SCD) is characterized by acute and chronic vaso-occlusion that can cause pain (1), acute chest syndrome (2), organ damage (3), stroke, and even death (4, 5). The pathogenic basis of “painful crisis” arising from vaso-occlusion in SCD is extremely complex (6-8). It is triggered by many factors including poor deformability of red blood cells (RBCs), adhesion among multiple cell types and blood components (e.g., sickle RBCs, endothelial cells, adherent leukocytes, and possibly platelets), as well as the local microenvironment (e.g., low oxygen concentration and acidosis). Under conditions of low oxygen partial pressure (pO₂), sickle RBCs experience intracellular sickle hemoglobin (HbS) polymerization, thereby reducing cell deformability (9). Such reductions in deformability can severely impact blood flow in narrow vessels, ultimately causing a transient or persistent blockage (10). Competition between the delay time for HbS polymerization and the RBC transit time in microcirculation is likely a key determinant of disease severity (11). Both in vitro (12) and ex vivo (13) models reveal that HbS polymerization and its effect on cellular rigidity play important roles in causing vascular obstruction. For example, HbS polymerization alone could be sufficient to cause complete RBC blockage in vasculature (12). Increases in microvascular transit time, arising from higher rigidity, of sickle RBCs cause peripheral vascular resistance to blood flow (13).

The search for better means to predict painful vaso-occlusion crises has focused on a range of hematological and rheological abnormalities. Significant correlations have been shown between pain rates and early death in patients with sickle cell anemia (14), and between early death and several risk factors such as fetal hemoglobin (HbF), hematocrit and white-cell count (15). However, factors such as patient age, sex, fetal hemoglobin (HbF) (16), intracellular HbS polymerization (17), or fraction of dense RBCs (18) do not appear to show a sufficiently direct correlation to the frequency and/or severity of pain crises. Although HbF level is generally considered important, its direct connection to disease severity is not fully established (19, 20). Some possible links between the incidence of painful crises and steady-state cell hydration (21) and/or deformability at isotonic osmolarity have been identified (22). Such connections, however, do not account for the observation that cell deformability and the proportion of dense cells vary longitudinally in the same patient during crisis (23). Changes have also been reported in the biorheological characteristics of sickle RBC suspension following deoxygenation in an in vitro vascular model (24).

### SUMMARY OF THE INVENTION

In some aspects, the invention is a high throughput method of measuring a property of an individual cell under controlled gas conditions, comprising: flowing a fluid comprising a plurality of cells through one or more constrictions; obtaining a measurement of an individual cell in the fluid; and regulating a level of gas in the fluid.

In some embodiments the property is a mechanical property. In other embodiments the property is deformability, rigidity, viscoelasticity, viscosity or adhesiveness.

In another embodiment the property is deformability. In some embodiments the property is a morphological property.

In other embodiments the property is cell shape. In some embodiments the cell shape is abnormal. In another embodiment the cell shape is round, disk shaped, biconcave, oblong, or sickle shaped.

In some embodiments of the invention the property is cell texture. In other embodiments the cell texture is abnormal. In another embodiment the cell texture is smooth, course or spiky.

In another embodiment the property is a kinetic, rheological or hematological property. In some embodiments the property is single cell velocity. In other embodiments the property is single cell capillary obstruction. In yet another embodiment the property is sickling, sphericity change, aspect ratio change, or change in cell texture.

In some embodiments the measurement is used to determine the fraction of obstructed cells. In other embodiments the measurement is used to determine the fraction of cells with an abnormal shape and/or texture. In another embodiment the measurement is used to determine the capillary obstruction ratio. In another embodiment the measurement is used to determine the delay time of an abnormal cell shape change. In yet another embodiment the measurement is used to determine the delay time of recovering from an abnormal cell shape change. In another embodiment the cell shape change is sickling.

In some embodiments the measurement is the distance traveled by one or more cells and/or the time to travel a certain distance through one or more constrictions at a certain pressure. In other embodiments the cells are from a subject. In another embodiment the cells are from a blood sample. In some embodiments cells comprise red blood cells, white blood cells, stem cells or epithelial cells. In some embodiments the cells are red blood cells.

In some embodiments the gas is selected from the group consisting of oxygen, nitrogen, carbon dioxide, nitric oxide, carbon monoxide, nitrous oxide, nitrogen dioxide and/or methane. In other embodiments the gas is oxygen. In another embodiment the level of the gas in the fluid is regulated to be at a concentration of less than 5%. In another embodiment the level of the gas in the fluid is regulated to be at a concentration from 5% to 20%. In some embodiments the level of the gas in the fluid is regulated to be at a concentration from 20% to 40%. In other embodiments the level of the gas in the fluid is regulated to be at a concentration from 40% to 60%. In other embodiments the level of the gas in the fluid is regulated to be greater than 60%. In another embodiment the level of the gas in the fluid is regulated to be at a concentration of about 20%. In other embodiments the level of the gas in the fluid is regulated to be at a concentration of about 5%. In some embodiment the level of the gas in the fluid is regulated to be at a concentration of about 2%. In other embodiments the level of the gas in the fluid is regulated to be at a concentration of about 20% oxygen, 5% carbon dioxide and about 75% nitrogen. In another embodiment the level of the gas in the fluid is regulated to be at a concentration of about 5% oxygen, 5% carbon dioxide and about 90% nitrogen. In yet another embodiment the level of the gas in the fluid is regulated to be at a concentration of about 2% oxygen, 5% carbon dioxide and about 93% nitrogen.

In some embodiments the property is measured at two or more different gas concentrations. In other embodiments the gas concentration is increased. In another embodiment the gas concentration is decreased. In yet another embodiment the property is measured as a function of time and as a function of gas concentration.

In another embodiment the cells are from a subject having or suspected of having a condition or disease selected from the group consisting of sickle cell disease (SCD), sickle cell trait (SCT), spherocytosis, ovalocytosis, alpha thalassemia, beta thalassemia, delta thalassemia, malaria, anemia, diabetes and leukemia. In some embodiments the cells are from a subject having or suspected of having sickle cell disease.

In some embodiments the fluid comprising the cells is flowed at a predetermined pressure gradient. In another embodiment the pressure gradient is in a range of about 0.10 Pa/μm to about 2.0 Pa/μm.

In other embodiments the fluid comprising the cells is flowed at a predetermined temperature. In another embodiment the temperature is a physiological temperature.

In other embodiments the fluid comprising a plurality of cells is flowed through the device of any of the below aspects and embodiments.

In some aspects the invention is a microfluidic device comprising: (a) a structure defining one or more microfluidic channels that each comprise (i) a first constriction having a first inlet orifice and a first outlet orifice, wherein the first inlet orifice is geometrically different from the first outlet orifice; and (b) a wall adjacent to the microfluidic channel, wherein at least a portion of the wall comprises a gas permeable membrane or film. In some embodiments the one or more microfluidic channels each also comprise (ii) a second constriction having a second inlet orifice and a second outlet orifice. In other embodiments the second inlet orifice is geometrically different from the second outlet orifice. In another embodiment the first inlet orifice is geometrically equal to the second inlet orifice and the first outlet orifice is geometrically equal to the second outlet orifice. In some embodiments the first constriction is arranged in series with the second constriction such that a flow path through the first constriction is longitudinally aligned with a flow path through the second constriction. In other embodiments the first constriction is arranged in series with the second constriction such that a flow path through the first constriction is non-longitudinally aligned with a flow path through the second constriction.

In some embodiments the one or more microfluidic channels further comprise a gap region between the first constriction and the second constriction. In other embodiments the gap region is of a length that allows one or more deformable objects to recover their shape after passing through the first constriction. In another embodiment the gap region is of a length that allows one or more deformable objects to partially recover their shape after passing through the first constriction. In some embodiments the gap region is of a length that does not allow one or more deformable to recover their shape after passing through the first constriction. In yet another embodiment the length of the gap region is equal to the length of the first constriction and/or the length of the second constriction.

In another embodiment the device further comprises a second constriction having a second inlet orifice and a second outlet orifice, and wherein the first and second constrictions are arranged in parallel such that a flow path through the first constriction is parallel with a flow path through the second constriction. In some embodiments the first constriction and/or the second constriction is a convergent conduit. In other embodiments each of the constrictions of the device is a convergent conduit. In another embodiment the first constriction and/or the second constriction is a divergent conduit.

In other embodiments each of the constrictions of the device is a divergent conduit. In another embodiment the constrictions of the device are a mix of convergent and divergent conduits.

In some embodiments the first inlet orifice and/or the first outlet orifice has a polygonal, curvilinear or circular shape. In other embodiments the polygonal shape is triangular. In other embodiments the second inlet orifice and/or the second outlet orifice has a polygonal, curvilinear or circular shape. In another embodiment the polygonal shape is triangular. In some embodiments the shape of the inlet orifice is two-dimensional. In other embodiments the shape of the inlet orifice is three-dimensional. In another embodiment the shape of the constriction is two-dimensional. In another embodiment the shape of the constriction is three-dimensional.

In some embodiments at least one dimension of the first inlet orifice and/or second inlet orifice is less than, greater than or equal to a dimension of the deformable object. In other embodiments the cross-sectional area of the at least one inlet orifice is less than, greater than, or equal to any select cross-sectional area of a deformable object. In another embodiment the first inlet orifice has a larger cross-sectional area than the first outlet orifice and/or the second inlet orifice has a larger cross-sectional area than the second outlet orifice. In another embodiment the first inlet orifice has a cross-sectional area in a range of 19 μm² to 23 μm² and the first outlet orifice has a cross-sectional area in a range of 10 μm² to 15 μm². In some embodiments the second inlet orifice has a cross-sectional area in a range of 19 μm² to 23 μm² and the second outlet orifice has a cross-sectional area in a range of 10 μm² to 15 μm².

In some embodiments the first inlet orifice has a smaller cross-sectional area than the first outlet orifice and/or the second inlet orifice has a smaller cross-sectional area than the first outlet orifice. In yet another embodiment the first inlet orifice has a cross-sectional area in a range of 10 μm² to 15 μm² and the first outlet orifice has a cross-sectional area in a range of 19 μm² to 23 μm². In another embodiment the second inlet orifice has a cross-sectional area in a range of 10 μm² to 15 μm² and the second outlet orifice has a cross-sectional area in a range of 19 μm² to 23 μm². In some embodiments the first constriction has a length in a range of 5 μm to 50 μm. In other embodiments the first constriction has a length in a range of 5 μm to 15 μm. In some embodiments the second constriction has a length in a range of 5 μm to 50 μm. In other embodiments the second constriction has a length in a range of 5 μm to 15 μm.

In some embodiments the microfluidic channel further comprises a substantially planar transparent wall that defines a surface of the first constriction and/or a surface of the second constriction. In other embodiments the substantially planar transparent wall comprises binding agents. In another embodiment the substantially planar transparent wall is glass or plastic. In other embodiments the substantially planar transparent wall has a thickness in a range of 0.05 mm to 0.1 mm. In another embodiment the substantially planar transparent wall permits observation into the microfluidic channel by microscopy. In another embodiment wherein at least one measurement of each deformable object that passes through one of the microfluidic channels can be obtained. In some embodiments the microfluidic channel has a height in a range of 1 μm to 10 μm.

In other embodiments the microfluidic channel has a height in a range of 3 μm to 5 μm.

In another embodiment the microfluidic channel has a height in a range of 0.5 μm to 3 μm.

In some embodiments the invention further comprises: a reservoir fluidically connected with the one or more microfluidic channels, and a pump that perfuses fluid from the reservoir through the one or more microfluidic channels. In some embodiments the reservoir further comprises a filter. In other embodiments the invention further comprises a microscope arranged to permit observation within the one or more microfluidic channels. In other embodiments at least one measurement of a cell that passes through one of the microfluidic channels can be obtained.

In some embodiments the invention further comprises a heat transfer element. In other embodiments the heat transfer element maintains the fluid at a predetermined temperature. In other embodiments the predetermined temperature is a physiologically relevant temperature.

In another embodiment the physiologically relevant temperature is in a range of 30° C. to 45° C. In another embodiment the physiologically relevant temperature is 37° C. In some embodiments the physiologically relevant temperature is 41° C.

In other embodiments of the invention the structure is a two-dimensional structure. In some embodiments the structure is a three-dimensional structure.

In other embodiments the invention further comprising a gas channel, wherein the gas channel contacts the gas permeable membrane or film. In some embodiments the gas channel contacts entire portion of the gas permeable membrane or film. In other embodiments the gas channel comprises an inlet. In yet another embodiment the gas channel comprises an outlet.

In another embodiment the gas permeable membrane or film is made of polydimethylsiloxane (PDMS), hydroxypropyl cellulose (HPC), hydroxypropyl methylcellulose (HPMC), cellulose triacetate (CTA), or poly(methyl methacrylate) (PMMA). In other embodiments the gas permeable membrane or film is made of polydimethylsiloxane (PDMS).

In some aspects the invention discloses a method for analyzing a condition or disease in a subject, the method comprising: (a) perfusing a fluid comprising one or more cells from the subject through the device of any one of claims A1-A64; (b) determining a property of one or more of the cells; and (c) comparing the property to an appropriate standard, wherein the results of the comparison are indicative of the status of the condition or disease in the subject. In some embodiments the method of analyzing the condition or disease is a method for detecting the presence or absence of the condition or disease in the subject, and wherein the property is indicative of the presence of the condition or disease in the subject. In other embodiments the method of analyzing the condition or disease is a method for detecting the presence or absence of the condition or disease in the subject, and wherein the property is indicative of the absence of the condition or disease in the subject. In yet another embodiment the method of analyzing the condition or disease is a method for determining the severity of a condition or disease in the subject, and wherein the property is indicative of the severity of the condition or disease in the subject. In some embodiments the method of analyzing the condition or disease is a method for predicting vaso-occlusion crises in a subject, and wherein the property is indicative of a likelihood that the subject will undergo vaso-occlusion crisis.

In other embodiments the cells comprise blood cells. In another embodiment of the invention the condition or disease is selected from the group consisting of sickle cell disease (SCD), sickle cell trait (SCT), spherocytosis, ovalocytosis, alpha thalassemia, beta thalassemia, delta thalassemia, malaria, anemia, diabetes and leukemia. In other embodiments the condition or disease is sickle cell disease.

In some embodiments the property is a mechanical property. In other embodiments the property is deformability, rigidity, viscoelasticity, viscosity or adhesiveness. In another embodiment the property is deformability. In another embodiment the property is a kinetic, rheological or hematological property. In some embodiments the property is single cell velocity. In other embodiments the property is single cell capillary obstruction. In yet another embodiment the property is cell sickling.

Other aspects of the invention include a method for monitoring the effectiveness of a therapeutic agent for treating a disease or condition in a subject comprising: (a) perfusing a fluid comprising one or more cells from the subject through the microfluidic device mentioned previously; (b) determining a property of one or more of the cells; (c) treating the subject with the therapeutic agent; and (d) repeating steps (a) and (b) at least once wherein a difference in the property of one or more cells is indicative of the effectiveness of the therapeutic agent.

Another aspect of the invention discloses a method for determining the effectiveness of a therapeutic comprising: (a) obtaining a biological sample from a subject comprising a cell; (b) perfusing a fluid comprising one or more cells from the subject through the microfluidic device mentioned previously; (c) determining a property of one or more of the cells; (d) contacting the biological sample comprising a cell with the therapeutic; (e) perfusing a fluid comprising the product of (d) through the microfluidic device mentioned previously; (f) determining a property of one or more of the cells from (e); and (g) comparing the property of one or more cells from (c) with the property of one or more cells from (f), wherein the results of the comparison are indicative of the effectiveness of the therapeutic.

In some embodiments the therapeutic is for treating sickle cell disease. In another embodiment the therapeutic is hydroxyurea (HU) or 5-hydroxymethylfurfural (Aes-103). In other aspects of the invention a real-time method for quantifying cell sickling and/or unsickling kinetics in response to varying levels of gas comprising: (a) perfusing a fluid comprising one or more blood cells through the microfluidic device mentioned previously, wherein the fluid has a first level of gas; (b) determining a property of one or more of the cells from (a); (c) perfusing a fluid comprising on or more cells through the microfluidic device mentioned previously; wherein the fluid has a second level of gas that is different from the first level; (d) determining a property of one or more of the cells from (c); and (e) quantifying the cell sickling and/or unsickling kinetics of the cells from (b) and (d) is disclosed.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A—Is a schematic of a microfluidic platform for investigation of biophysical alterations in sickle red blood cells (RBCs) under transient hypoxia conditions. The schematic of the microfluidic device with O₂ control may be used for studying kinetics of cell sickling and unsickling and identification of cell sickling events from a microscopic image (arrows indicate the sickled RBCs).

FIG. 1B—Is a schematic of a microfluidic device with capillary-inspired structures for single cell rheology study. Note that schematics are not drawn to scale.

FIG. 2A—Profiles of cell sickling and unsickling under transient hypoxia conditions with 2% for lowest O2 concentration. This profile of short-term transient DeOxy (O₂ concentration less than 5% O2 for ˜25 s).

FIG. 2B—Profiles of cell sickling and unsickling under transient hypoxia conditions with 2% for lowest O₂ concentration. This profile is of long-term DeOxy (O₂ concentration less than 5% O₂ for 220 s).

FIG. 2C—Profiles of cell sickling and unsickling under transient hypoxia conditions with 2% for lowest O₂ concentration. These profiles are of sickled fraction of multiple sickle cell disease (SCD) samples during the long-term transient DeOxy (each curve represents an individual patient sample).

FIG. 3A—Kinetics of cell sickling: delay time of cell sickling for 5% sickled fraction. Arrows indicate severe cases defined as those where sickling delay time was less than 25 s. Open circles represent on hydroxyurea therapy (on-HU) and filled circles represent off hydroxyurea therapy (off-HU).

FIG. 3B—Kinetics of cell sickling: delay time of cell sickling for 10% sickled fraction. Arrows indicate severe cases defined as those where sickling delay time was less than 25 s. Open circles represent on-HU and filled circles represent off-HU.

FIG. 3C—Distributions of maximum sickled fractions under short-term transient DeOxy state (O₂ concentration less than 5% for 25 s). Arrows indicate severe cases defined as those where sickling delay time was less than 25 s. Open circles represent on-HU and filled circles represent off-HU.

FIG. 3D—Distributions of maximum sickled fractions under long-term transient DeOxy state (O₂ concentration less than 5% for 220 s).

FIG. 4A—Individual sickle RBC rheology under transient hypoxia. Time sequence of RBCs traveling through capillary-inspired structures. Arrows indicate sickled cells that are unable to pass through the micro-gates thereby obstructing RBC flow.

FIG. 4B—Representative velocity profile of RBC flow with each data point representing the average speed of an individual RBC travelling through five of the periodic micro-gates under a pressure difference of 15 ml water in a 60 ml Terumo plastic syringe tube (equivalent to 22.6 mm H₂O). The shaded area indicates an O₂ concentration lower than 5%.

FIG. 4C—Cell capillary obstruction ratio as a function of % sickle hemoglobin (HbS). The arrow indicates a severe case with the highest capillary obstruction ratio.

FIG. 5A—Shows the role of cell density on delay time of cell sickling under the short-term DeOxy state.

FIG. 5B—Shows the role of cell density on sickled fraction under the short-term DeOxy state.

FIG. 6A—Delay time of cell sickling for maximum sickled fraction of individual samples under long-term DeOxy state.

FIG. 6B—Delay time of cell unsickling for maximum sickled fraction of individual samples under long-term DeOxy state.

FIG. 7A—Velocity distribution of deformable sickle RBCs: cell velocity against mean corpuscular volume (MCV) under the Oxy state.

FIG. 7B—Velocity distribution of deformable sickle RBCs: cell velocity against patient's HU status and transfusion under the Oxy and DeOxy states.

FIG. 8A—Density distribution among four density populations.

FIG. 8B—Profiles of sickled fractions for a representative on-HU case under short-term DeOxy state.

FIG. 8C—Profiles of sickled fractions for a representative off-HU case under short-term DeOxy state.

FIG. 8D—Profiles of sickled fractions for representative on-HU case under long-term DeOxy state.

FIG. 8E—Profiles of sickled fractions for representative off-HU case under long-term DeOxy state.

FIG. 9A—Role of cell density on delay time of cell unsickling under the long-term DeOxy states.

FIG. 9B—Role of cell density on sickled fraction under the long-term DeOxy states.

FIG. 10A—Effects of % HbF on kinetics of cell sickling of density-fractionated populations. Delay time of cell sickling under short-term DeOxy state.

FIG. 10B—Effects of % HbF on kinetics of cell sickling of density-fractionated populations. Delay time of sickled fraction under short-term DeOxy state.

FIG. 11A—Distribution of Hb types in density-separated populations in all samples (n=13).

FIG. 11B—Distribution of Hb types in density-separated populations in off-HU samples (n=5).

FIG. 11C—Distribution of Hb types in density-separated populations in on-HU samples (n=8).

FIG. 11D—Distributions of mean intracellular HbF concentration (MCHC-F) and mean intracellular HbS concentration (MCHC-S) in density-separated populations.

FIG. 12—Effects of the Aes-103 concentration on the sickled fraction under long-term DeOxy state.

FIG. 13A—Relationships between the effective sickled fraction and intracellular hemoglobin concentrations of MCHC-F. Solid circles represent all RBCs and empty circles represent density-fractionated RBCs.

FIG. 13B—Relationships between the effective sickled fraction and intracellular hemoglobin concentrations of MCHC-S. Solid circles represent all RBCs and empty circles represent density-fractionated RBCs.

FIG. 14A—Identification of cell sickling from a microscopic image (arrows indicate the sickled RBCs).

FIG. 14B—Sickled fraction as a function of Aes-103 concentration.

FIG. 14C—Variation in response among different on-HU and off-HU patient samples.

FIG. 15A—Shows continuing DeOxy and ReOxy cycles.

FIG. 15B—Shows in vitro hypoxia-induced cell sickling, tracking a single RBC sickling to unsickling during one cycle of transient hypoxia.

FIG. 16A—Shows randomness in hypoxia-induced cellular morphological sickling during continuing DeOxy and ReOxy cycles. Initiation sites of cell transformation are highlighted with arrows that do not point directly up, indicating the primary sites for intracellular HbS polymerization, with respect to the orientation of individually tracked sickle RBCs highlighted with arrows pointing directly up.

FIG. 16B—Shows heterogeneous cell deformity for individual sickled cells with initially biconcave and permanently sickled shapes.

FIG. 17A—Shows representative curves of kinetics of cell sickling in a sickled fraction during continuing DeOxy cycles. Error bars indicate standard deviations.

FIG. 17B—Shows representative curves of kinetics of delay time of cell sickling during continuing DeOxy cycles. Error bars indicate standard deviations.

FIG. 18A—Shows normalized kinetics of cell sickling as functions of DeOxy cycle for a sickled fraction. Each symbol represents the mean value for an individual patient sample. The filled circles represent the average value of six patient samples and dashed curves are the corresponding power law interpolations. Error bars indicate standard deviations.

FIG. 18B—Shows normalized kinetics of cell sickling as functions of DeOxy cycle for delay time of cell sickling. Each symbol represents the mean value for an individual patient sample. The filled circles represent the average value of six patient samples and dashed curves are the corresponding power law interpolations. Error bars indicate standard deviations.

FIG. 19 Shows time for completion of cell sickling as a function of delay time for 134 individual sickle RBCs during the first DeOxy cycle and the fifth DeOxy cycle. Resolution of time is one second.

### DETAILED DESCRIPTION OF CERTAIN EMBODIMENTS OF THE INVENTION

Described herein are devices and methods for assessing cell properties under controlled gas environments. Accordingly, a microfluidics-based model was developed to quantify cell-level processes modulating the pathophysiology of disease (e.g., sickle cell disease (SCD), spherocytosis, ovalocytosis, alpha thalassemia, beta thalassemia, delta thalassemia, malaria an anemia). This in vitro model enabled quantitative investigations of the kinetics of cell processes and transformations such as cell sickling, unsickling and cell rheology. Examples of the use of the devices of the invention are included in the Examples below. The Examples use SCD to demonstrate the effectiveness of the device and methods described herein. However, the invention is not limited to SCD. Briefly, in the Examples, short-term and long-term hypoxia conditions were created to simulate normal and retarded transit scenarios in microvasculature. Using blood samples from 25 SCD patients with sickle hemoglobin (HbS) levels varying from 64% to 90.1%, cell biophysical alterations were investigated during blood flow correlated with hematological parameters, HbS level and hydroxyurea therapy. From these measurements, two severe cases of SCD were identified that were also independently validated as severe from a genotype-based disease severity classification. These results point to the use of this method as a diagnostic indicator of disease severity. In addition, the role of cell density in the kinetics of cell sickling was investigated. An effect of HU therapy was observed mainly in relatively denser cell populations, and sickled fraction increased with cell density. These results support the use of the microfluidic platform described as a unique and quantitative approach to assess the kinetic, rheological and hematological factors involved in vaso-occlusive events associated with disease and to develop alternative diagnostic tools of disease severity. Such insights may also lead to a better understanding of the pathogenic basis and mechanism of drug response in disease.

### Microfluidic Devices

Devices are provided herein for evaluating, characterizing, and assessing properties of cells under controlled gas conditions. In particular, devices are provided for measuring, evaluating and characterizing dynamic mechanical responses of biological cells, e.g., red blood cells, to changes in the level of a gas (e.g., oxygen). The devices are typically designed and configured to permit measurements of cell deformability in a high throughput manner.

In some cases, the devices are designed and configured to permit microscopic measurements, e.g., fluorescence measurements, on cells passing through the device. The devices, in some examples, are designed and configured to create low Reynolds number fluid regimes. Such fluid regimes are useful for evaluating the effects of constriction entrance architecture (e.g., inlet orifice size and/or shape) on the sensitivity of cell deformability measurements.

The devices typically include a structure defining one or more microfluidic channels through which a fluid that comprises one or more cells may pass. When the structure defines two or more microfluidic channels, typically each of the channels is at least partially fluidically isolated from the other(s).

Each of the one or more microfluidic channels typically contains one or more of constrictions (e.g., two or three-dimensional). As used herein, the term “constriction” refers to a relatively narrow portion of a fluid passage, having an inlet orifice and an outlet orifice. As used herein, the term “inlet orifice” refers to an opening that defines an entrance into a narrow portion of a fluid passage and the term “outlet orifice” refers to an opening that defines an exit from a narrow portion of a fluid passage. Between an inlet orifice and outlet orifice, the constriction comprises a “conduit” through which a fluid and/or object may pass.

The inlet orifices and outlet orifices can have any of variety of shapes, including, for example, polygonal (e.g., triangular, rectangular), curvilinear or circular shape. In one example, the shape of the at least one inlet/outlet orifice is two-dimensional. In another example, it is three-dimensional. In either case, one or more dimensions of the at least one inlet orifice is less than, greater than, or equal to a dimension of a cell.

An inlet orifice may have a cross-sectional area of up to 0.1 μm², 0.5 μm², 1 μm², 2 μm², 3 μm², 4 μm², 5 μm², 6 μm², 7 μm², 8 μm², 9 μm², 10 μm², 11 μm², 12 μm², 13 μm², 14 μm², 15 μm², 16 μm², 17 μm², 18 μm², 19 μm², 20 μm², 21 μm², 22 μm², 23 μm², 24 μm², 25 μm², 26 μm², 27 μm², 28 μm², 29 μm², 30 μm², 31 μm², 32 μm², 33 μm², 34 μm², 35 μm², 36 μm², 37 μm², 38 μm², 39 μm², 40 μm², 41 μm², 42 μm², 43 μm², 44 μm², 45 μm², 46 μm², 47 μm², 48 μm², 49 μm², 50 μm², 55 μm², 60 μm², 65 μm², 70 μm², 75 μm², 80 μm², 85 μm², 90 μm², 95 μm², 100 μm², 150 μm², 200 μm², 250 μm², or more. An inlet orifice may have a cross-sectional area in a range of 0.1 μm² to 1 μm², 1 μm² to 2 μm², 1 μm² to 10 μm², 2 μm² to 5 μm², 5 μm² to 10 μm², 5 μm² to 50 μm², 10 μm² to 15 μm², 15 μm² to 20 μm², 20 μm² to 30 μm², 30 μm to 40 μm², 40 μm² to 50 μm², 50 μm² to 100 μm², or 100 μm² to 200 μm², for example.

In some embodiments, the inlet orifice is at least 1 μm wide, at least 2 μm wide, at least 3 μm wide, at least 4 μm wide, at least 5 μm wide, at least 6 μm wide, at least 8 μm wide, at least 10 μm wide, at least 15 μm wide or at least 20 μm wide. In other embodiments, the inlet orifice is at least 1 μm in height, at least 2 μm in height, at least 3 μm in height, at least 4 μm in height, at least 5 μm in height, at least 6 μm in height, at least 8 μm in height, at least 10 μm in height, at least 15 μm in height or at least 20 μm in height. In one specific embodiment, the inlet orifice is 4 μm wide and 5 μm in height.

An outlet orifice may have a cross-sectional area of up to 0.1 μm², 0.5 μm², 1 μm², 2 μm², 3 μm², 4 μm², 5 μm², 6 μm², 7 μm², 8 μm², 9 μm², 1O μm², 11 μm², 12 μm², 13 μm², 14 μm², 15 μm2, 16 μm², 17 μm², 18 μm², 19 μm², 2O μm², 21 μm², 22 μm², 23 μm², 24 μm², 25 μm², 26 μm², 27 μm2, 28 μm², 29 μm², 3O μm², 31 μm², 32 μm², 33 μm², 34 μm², 35 μm², 36 μm², 37 μm², 38 μm², 39 μm², 40 μm², 41 μm², 42 μm², 43 μm², 44 μm², 45 μm², 46 μm², 47 μm², 48 μm², 49 μm², 50 μm², 55 μm², 60 μm², 65 μm², 70 μm², 75 μm², 80 μm², 85 μm², 90 μm², 95 μm², 100 μm², 150 μm², 200 μm², 250 μm², or more.

An outlet orifice may have a cross-sectional area in a range of 0.1 μm² to 1 μm², 1 μm² to 2 μm², 1 μm² to 10 μm², 2 μm² to 5 μm², 5 μm² to 10 μm², 5 μm² to 50 μm², 10 μm² to 15 μm², 15 μm² to 20 μm², 20 μm² to 30 μm², 30 μm² to 40 μm², 40 μm² to 50 μm², 50 μm² to 100 μm², or 100 μm² to 200 μm², for example.

In some embodiments, the outlet orifice is at least 1 μm wide, at least 2 μm wide, at least 3 μm wide, at least 4 μm wide, at least 5 μm wide, at least 6 μm wide, at least 8 μm wide, at least 10 μm wide, at least 15 μm wide or at least 20 μm wide. In other embodiments, the outlet orifice is at least 1 μm in height, at least 2 μm in height, at least 3 μm in height, at least 4 μm in height, at least 5 μm in height, at least 6 μm in height, at least 8 μm in height, at least 10 μm in height, at least 15 μm in height or at least 20 μm in height. In one specific embodiment, the outlet orifice is 4 μm wide and 5 μm in height. The geometry, e.g., size and shape, of the inlet and outlet orifices may or may not be the same. In some cases, the inlet orifice of at least one of the constrictions is geometrically different from the outlet orifice of the same constriction. As used herein, the term “geometrically different” means different in size and/or shape. For example, the inlet orifice(s) in one or more of the constrictions can have a larger cross-sectional area than the outlet orifice(s) in the same constriction(s), e.g., 19 μm² to 23 μm² versus 10 μm² to 15 μm². Alternatively, the inlet orifice(s) has a smaller cross-sectional area than the outlet orifice(s) in the same constriction, e.g., 10 μm² to 15 μm² versus 19 μm² to 23 μm².

The difference between the cross-sectional area of an inlet orifice and the cross-sectional area of an outlet orifice may be up to 0.1 μm², 0.5 μm², 1 μm², 2 μm², 3 μm², 4 μm², 5 μm², 6 μm², 7 μm², 8 μm², 9 μm², 10 μm², 11 μm², 12 μm², 13 μm², 14 μm², 15 μm², 16 μm², 17 μm², 18 μm², 19 μm², 20 μm², 21 μm², 22 μm², 23 μm², 24 μm², 25 μm², 26 μm², 27 μm², 28 μm², 29 μm², 30 μm², 31 μm², 32 μm², 33 μm², 34 μm², 35 μm², 36 μm², 37 μm², 38 μm², 39 μm², 40 μm², 41 μm², 42 μm², 43 μm², 44 μm², 45 μm², 46 μm², 47 μm², 48 μm², 49 μm², 50 μm², 55 μm², 60 μm², 65 μm², 70 μm², 75 μm², 80 μm², 85 μm², 90 μm², 95 μm², 100 μm², or more.

The difference between the cross-sectional area of an inlet orifice and the cross-sectional area of an outlet orifice may be in a range of 0.1 μm² to 1 μm², 1 μm² to 2 μm², 1 μm² to 10 μm², 2 μm² to 5 μm², 5 μm² to 10 μm², 5 μm² to 50 μm², 10 μm² to 15 μm², 15 μm² to 20 μm², 20 μm² to 30 μm², 30 μm² to 40 μm², 40 μm² to 50 μm², or 50 μm² to 100 μm², for example.

The one or more constrictions can have a conduit length (distance between inlet orifice and outlet orifice) of up to 0.1 μm, 0.5 μm, 1 μm, 2 μm, 3 μm, 4 μm, 5 μm, 6 μm, 7 lam, 8 μm, 9 μm, 10 μm, 11 μm, 12 μm, 13 μm, 14 μm, 15 μm, 16 μm, 17 μm, 18 μm, 19 lam, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 lam, 44 μm, 45 μm, 46 μm, 47 μm, 48 μm, 49 μm, 50 μm, 55 μm, 60 μm, 65 μm, 70 μm, 75 lam, 80 μm, 85 μm, 90 μm, 95 μm, 100 μm, 150 μm, 200 μm, 250 μm, 300 μm, 350 μm, 400 μm, 450 μm, 500 μm, 1 mm or more. In one specific embodiment, the conduit length is 15 μm.

The one or more constrictions can have a conduit length (distance between inlet orifice and outlet orifice) in a range of 0.1 μm to 1 μm, 1 μm to 10 μm, 5 μm to 50 μm, 25 μm to 100 μm, 50 μm to 200 μm, 150 μm to 500 μm, or 500 μm to 1 mm.

The one or more constrictions may have an average cross-sectional area, perpendicular to the flow direction through its conduit, of up to 0.1 μm², 0.5 μm², 1 μm², 2 μm², 3 μm², 4 μm², 5 μm², 6 μm², 7 μm², 8 μm², 9 μm², 10 μm², 11 μm², 12 μm², 13 μm², 14 μm², 15 μm², 16 μm², 17 μm², 18 μm², 19 μm², 20 μm², 21 μm², 22 μm², 23 μm², 24 μm², 25 μm², 26 μm², 27 μm2, 28 μm², 29 μm², 30 μm², 31 μm², 32 μm², 33 μm², 34 μm², 35 μm², 36 μm², 37 μm², 38 μm², 39 μm², 40 μm², 41 μm², 42 μm², 43 μm², 44 μm², 45 μm², 46 μm², 47 μm², 48 μm², 49 μm², 50 μm², 55 μm², 60 μm², 65 μm², 70 μm², 75 μm², 80 μm², 85 μm², 90 μm², 95 μm², 100 μm², 150 μm², 200 μm², 250 μm², or more.

The one or more constrictions may have an average cross-sectional area, perpendicular to the flow direction through its conduit, in a range of 0.1 μm² to 1 μm², 1 μm² to 2 μm², 1 μm² to 10 μm², 2 μm² to 5 μm², 5 μm² to 10 μm², 5 μm² to 50 μm², 10 μm² to 15 μm², 15 μm² to 20 μm², 20 μm² to 30 μm², 30 μm² to 40 μm², 40 μm² to 50 μm², 50 μm² to 100 μm², or 100 μm² to 200 μm², for example.

In some embodiments the one or more constrictions have a cross-sectional width of at least 1 μm, at least 2 μm, at least 3 μm, at least 4 μm, at least 5 μm, at least 6 μm, at least 8 pm, at least 10 μm, at least 15 μm or at least 20 μm. In other embodiments the one or more constrictions have a cross-sectional height of at least 1 μm, at least 2 μm, at least 3 μm, at least 4 μm, at least 5 μm, at least 6 μm, at least 8 μm, at least 10 μm, at least 15 μm or at least 20 μm. In one specific embodiment, the one or more constrictions have a cross-sectional width of 8 μm and a cross-sectional height of 5 μm.

The one or more constrictions may define a convergent conduit. The one or more constrictions may define a conduit having a cross-sectional area, perpendicular to the flow direction through the conduit, that converges (narrows) at a rate of 0.001 μm²/μm, 0.01 μm²/μm, 0.05 μm²/μm, 0.1 μm²/μm, 0.2 μm²/μm, 0.3 μm²/μm, 0.4 μm²/μm, 0.5 μm²/μm, 0.6 μm²/μm, 0.7 μm²/μm, 0.8 μm²/μm, 0.9 μm²/μm, 1 μm²/μm, 2 μm²/μm, 5 μm²/μm, 10 μm²/μm, or more.

The one or more constrictions may define a conduit having a cross-sectional area, perpendicular to the flow direction through the conduit, that converges at a rate in a range of 0.001 μm²/μm to 0.01 μm²/μm, 0.01 μm²/μm to 0.1 μm²/μm, 0.1 μm²/μm to 0.5 μm²/μm, 0.1 μm²/μm to 1 μm²/μm, or 1 μm²/μm to 10 μm²/μm, or more.

The one or more constrictions may define a divergent conduit. The one or more constrictions may define a conduit having a cross-sectional area, perpendicular to the flow direction through the conduit, that diverges (widens) at a rate of 0.001 μm²/μm, 0.01 μm²/μm, 0.05 μm²/μm, 0.1 μm²/μm, 0.2 μm²/μm, 0.3 μm²/μm, 0.4 μm²/μm, 0.5 μm²/μm, 0.6 μm²/μm, 0.7 μm²/μm, 0.8 μm²/μm, 0.9 μm²/μm, 1 μm²/μm, 2 μm²/μm, 5 μm²/μm, 10 μm²/μm, or more.

The one or more constrictions may define a conduit having a cross-sectional area, perpendicular to the flow direction through the conduit, that diverges at a rate in a range of 0.001 μm²/μm to 0.01 μm²/μm, 0.01 μm²/μm to 0.1 μm²/μm, 0.1 μm²/μm to 0.5 μm²/μm, 0.1 μm²/μm to 1 μm²/μm, or 1 μm²/μm to 10 μm²/μm, or more.

Other non-uniform conduit geometries are envisioned. For example, a constriction may have a conduit with an undulating, wavy, jagged, irregular or randomly altering cross-sectional area along its length.

The one or more microfluidic channels in the device described herein, when each contains at least two constrictions, can further contain a gap region between each successive constriction. In one example, this gap region is of a length that allows one or more deformable objects (e.g., cells, vesicles, biomolecular aggregates, platelets or particles) to recover, at least partially, their shape after passing through the first constriction (e.g., equal to the length of one of the constrictions and/or the length of its successive constriction). In another example, the gap region is of a length that does not allow one or more cells to recover their shape after passing through each constriction.

The gap region may have a length (e.g., distance between outlet orifice of a first constriction and an inlet orifice of a second constriction, aligned in series) of up to 0.1 μm, 0.5 μm, 1 μm, 2 μm, 3 μm, 4 μm, 5 μm, 6 μm, 7 μm, 8 μm, 9 μm, 10 μm, 11 μm, 12 μm, 13 pm, 14 μm, 15 μm, 16 μm, 17 μm, 18 μm, 19 μm, 20 μm, 21 μm, 22 μm, 23 μm, 24 μm, 25 pm, 26 μm, 27 μm, 28 μm, 29 μm, 30 μm, 31 μm, 32 μm, 33 μm, 34 μm, 35 μm, 36 μm, 37 pm, 38 μm, 39 μm, 40 μm, 41 μm, 42 μm, 43 μm, 44 μm, 45 μm, 46 μm, 47 μm, 48 μm, 49 pm, 50 μm, 55 μm, 60 μm, 65 μm, 70 μm, 75 μm, 80 μm, 85 μm, 90 μm, 95 μm, 100 μm, 150 μm, 200 μm, 250 μm, 300 μm, 350 μm, 400 μm, 450 μm, 500 μm, 1 mm or more. In a specific embodiment, the gap region has a length of 15 μm.

The gap region may have a length in a range of 0.1 μm to 1 μm, 1 μm to 10 μm, 5 μm to 50 μm, 25 μm to 100 μm, 50 μm to 200 μm, 150 μm to 500 μm, or 500 μm to 1 mm.

In one example, the one or more microfluidic channels each comprise at least two constrictions: (a) a first constriction having a first inlet orifice and a first outlet orifice, and (b) a second constriction having a second inlet orifice and a second outlet orifice. The first constriction and the second constrictions can be arranged in parallel such that a flow path through one constriction is parallel with a flow path through the other constriction. The first constriction and the second constriction can be arranged in series such that a flow path through one constriction is parallel with a flow path through the other constriction. The first constriction and the second constriction can be arranged in series such that a flow path through one constriction is parallel with a flow path through the other constriction. In these examples, the first inlet orifice and the first outlet orifice may be geometrically equal to or geometrically different than the second inlet orifice and the second outlet orifice, respectively.

In another example, the one or more microfluidic channels in the device each contain a plurality of constrictions arranged in series, each constriction of the plurality being a non-uniform conduit. In both examples described above, the constrictions can be arranged in series such that a flow path through each of the constrictions is aligned, longitudinally or non-longitudinally, with a flow path through each other constriction(s). Moreover, one, more than one, or all of the constrictions in the series may be a non-uniform conduit, e.g., a convergent conduit or a divergent conduit.

When a device contains at least two microfluidic channels, the constrictions in one of the channels can be arranged in parallel with those in each other channel(s) such that a flow path through the former is parallel with a flow path through the latter. Devices containing at least two microfluidic channels, may be designed and constructed such that the resistance to flow through each channel is different. Alternatively, devices containing at least two microfluidic channels, may be designed and constructed such that the resistance to flow through each channel is essentially the same.

Furthermore, when a device contains at least two microfluidic channels, the fluidics associated the channels can be arranged such that flow through each channel(s) travels in the same direction, or in opposite directions. When a device contains at least two microfluidic channels and the fluidics associated the channels are arranged such that flow through each channel(s) travels in the same direction, the channels are typically either partially fluidically isolated or fluidically isolated. When a device contains at least two microfluidic channels and the fluidics associated the channels are arranged such that flow through each channel(s) travels in opposite directions, the channels are typically fluidically isolated. Channels that are “fluidically isolated” are configured and designed such that there is no fluid exchanged directly between the channels. Channels that are “partially fluidically isolated” are configured and designed such that there is partial (e.g., incidental) fluid exchanged directly between the channels.

Devices containing one or more microfluidic channels further contain a wall adjacent to the microfluidic channel where at least a portion of the wall is gas permeable. As used herein, “adjacent to” refers to a physical proximity to the channel such that at least a portion of the wall and at least a portion of the channel are in physical contact or are separated by a space that contains the gas. “Adjacent to” could mean that the wall defines a surface of at least one of the constrictions. Adjacent to could also mean that the wall defines an inner surface and/or outer surface of the microfluidic device. For example the microfluidic channel may have a top surface, bottom surface, side surface or end surface that contacts and/or contains a fluid that is flowed through one or more of the microfluidic channels. This gas permeable portion of the wall, which can be, for example a gas permeable membrane or film e.g., polydimethylsiloxane (PDMS), permits the control of the level of a gas in the microfluidic device. In some embodiments, the gas permeable film has a thickness ranging from 5 μm to 500 μm. In some embodiments , the gas permeable film has a thickness ranging from 5 μm to 20 μm, from 5 μm to 50 μm, from 5 μm to 100 μm, from 5 μm to 150 μm, from 5 μm to 200 μm, from 5 μm to 250 μm, from 5 μm to 300 μm, from 5 μm to 400 μm, from 5 μm to 500 μm, from 50 μm to 100 μm, from 50 μm to 150 μm, from 50 μm to 200 μm, from 50 μm to 300 μm, from 50 μm to 400 μm, from 50 μm to 500 μm, from 100 μm to 200 μm, from 100 μm to 300 μm, from 100 μm to 400 μm, from 100 μm to 500 μm, from 200 μm to 300 μm, from 200 μm to 400 μm, from 200 μm to 500 μm, from 300 μm to 400 μm, from 300 μm to 500 μm or from 400 μm to 500 μm. As one specific example, the gas permeable film has a thickness of about 150 μm. It should be appreciated that the gas permeable membrane or film may make up an entire wall or a portion of a wall of the microfluidic channel. In some embodiments the gas permeable membrane makes up from 1% to 100% of the surface area of a wall of the device. In some embodiments, the gas permeable membrane makes up from 1% to 5%, from 1% to 10%, from 1% to 20%, from 1% to 30%, from 1% to 50%, from 1% to 60%, from 1% to 80%, from 1% to 100%, from 5% to 10%, from 5% to 20%, from 5% to 30%, from 5% to 50%, from 5% to 60%, from 5% to 80%, from 5% to 100%, from 20% to 30%, from 20% to 50%, from 20% to 60%, from 20% to 80%, from 20% to 100%, from 30% to 50%, from 30% to 60%, from 30% to 80%, from 30% to 100%, from 50% to 60%, from 50% to 80%, from 50% to 100%, or from 80% to 100% of a wall of the microfluidic device. It should be appreciated that one or more walls of the microfluidic device may have at least a portion of the wall that is made of a gas permeable membrane or film.

The gas permeable membrane or film may be permeable to any number of gases that are supplied to the gas permeable membrane or film. For example the membrane or film may be permeable to gasses including but not limited to oxygen, nitrogen, carbon dioxide, nitric oxide, carbon monoxide, nitrous oxide, nitrogen dioxide and/or methane. In a particular embodiment, the membrane or film is permeable to oxygen. The gas permeable membrane or film may be constructed of any suitable material that is permeable to any of the gases, described herein. For example the gas permeable membrane or film may be made of a material including but not limited to polydimethylsiloxane (PDMS), hydroxypropyl cellulose (HPC), hydroxypropyl methylcellulose (HPMC), cellulose triacetate (CTA), or poly(methyl methacrylate) (PMMA). Other gas permeable membranes or films are known in the art, such as those disclosed in Budd et al. (Peter M. Budd and Neil B. McKeown, Highly permeable polymers for gas separation membranes, Polym. Chem., 2010,1, 63-68; the entire contents of which are hereby incorporated by reference). In a specific embodiment, the gas permeable film is made of PDMS.

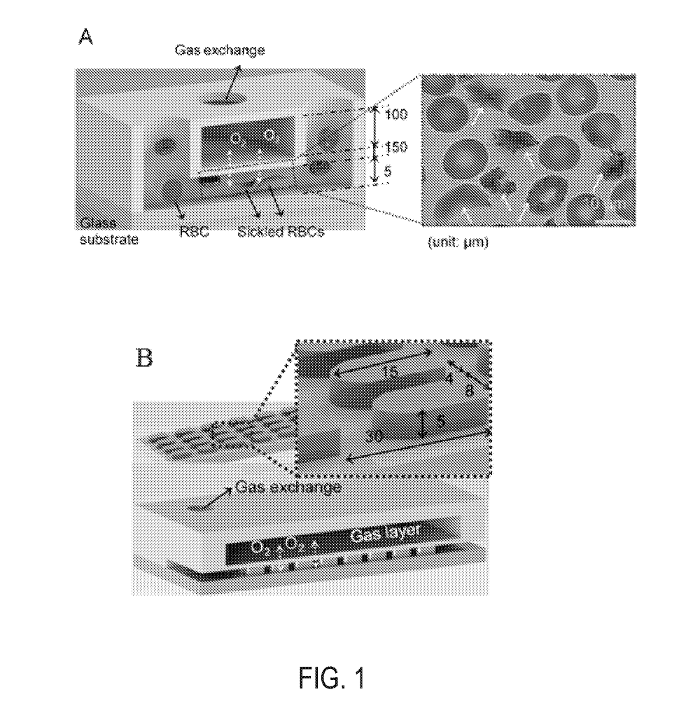

Any of the devices, described herein, may also contain a gas channel. This gas channel may be used to supply a gas to the gas permeable membrane or film of the device in order to regulate the gas content of the fluid in the device. The gas channel may encase the gas permeable membrane or film on a wall of the microfluidic channel such that gas exchange can occur between the gas in the gas channel and the fluid in the microfluidic channel through the gas permeable membrane or film. An exemplary microfluidic device with a gas channel encasing a gas permeable layer is shown in FIG. 1A-1B. The gas channel is separated from the microfluidic channel by a gas permeable membrane to allow gas exchange between the gas channel and a fluid in the microfluidic device. The gas channel may be any size or shape suitable for supplying a gas to the gas permeable membrane or film of the microfluidic device. In some embodiments, the gas channel is between 10 μm and 10 mm in height. In one specific embodiment, the gas channel is 100 μm in height. It should also be appreciated that any of the microfluidic devices may comprise one or more gas channels to deliver one or more gasses to any portion of the microfluidic device with a gas permeable membrane or film.

In some embodiments, the gas channel comprises at least one inlet and/or at least one outlet. A gas or gas mixture may be supplied to the inlet of the gas channel from one or more tanks containing the gas or gas mixture. In some non-limiting embodiments, the gas supplied to the gas channel is oxygen, nitrogen, carbon dioxide, nitric oxide, carbon monoxide, nitrous oxide, nitrogen dioxide, methane, or any combination thereof. In some embodiments the gas supplied to the gas channel contains oxygen. In some embodiments the gas supplied contains between 1% and 100% oxygen. In some embodiments the gas supplied contains from 1% to 2%, from 1% to 3%, from 1% to 5%, from 1% to 10%, from 1% to 20%, from 1% to 40%, from 1% to 60%, from 1% to 80%, from 1% to 100%, from 5% to 10%, from 5% to 20%, from 5% to 40%, from 5% to 60%, from 5% to 80%, from 5% to 100%, from 20% to 40%, from 20% to 60%, from 20% to 80%, from 20% to 100%, from 40% to 60%, from 40% to 80%, from 40% to 100%, from 60% to 80%, from 60% to 100% or from 80% to 100% oxygen. In some embodiments the gas supplied to the gas channel contains about 2%, about 5%, or about 20% oxygen. As used herein, the term “about,” or “approximately” as applied to one or more values of interest, refers to a value that is similar to a stated reference value. In certain embodiments, the term “approximately” or “about” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (for example, when such number would exceed 100% of a possible value). In some embodiments the gas supplied to the gas channel contains about 20% oxygen, 5% carbon dioxide and about 75% nitrogen. In other embodiments the gas supplied to the gas channel contains about 5% oxygen, 5% carbon dioxide and about 90% nitrogen. In yet other embodiments the gas supplied to the gas channel contains about 2% oxygen, 5% carbon dioxide and about 93% nitrogen. The gas or gas mixture may be supplied to one or more inlets of one or more gas channels using any suitable means, such as tubing or hoses.

The gas or gas mixture may be delivered to the gas channel continuously such that the gas enters the inlet of the gas channel and exits from the outlet of the gas channel. This ensures that the gas or gas mixture in the channel remains consistent as gas exchanges across the gas permeable membrane. As used herein, consistent means that the level of, or % composition of a gas in a given space (e.g., a channel) does not vary by a large amount. In some embodiments consistent means that the level of, or % composition of a gas entering the gas channel does not vary by more than 1%, 2%, 3%, 4%, 5%, 8% or 10% before exiting the gas channel. The gas or gas mixture may be flowed through the gas channel at any suitable rate. The gas in the gas channel may regulated at a specific pressure. In some embodiments the pressure of the gas in the gas chamber is from 1 psi to 10 psi. In a specific embodiment, the pressure of the gas in the gas chamber is regulated to be about 5 psi.

The device may be configured such that the gas or gas mixture, supplied to one or more gas inlets of the device, can be switched to a different gas or gas mixture. This enables the device to dynamically control the gas content of a fluid in the microfluidic channel. For example, a fluid containing cells flowing through one or more microfluidic channels of the device can be exposed to a gas with high oxygen content (e.g., 20% oxygen) for a given time through the gas channel. As the fluid containing cells flows through the one or more microfluidic channels, a different gas may be supplied to the same gas channel or a different gas channel. For example the gas delivered to the gas channel can be switched to a gas with low oxygen content (e.g., 2% oxygen). This allows for the dynamic observation/measurement of cell parameters in response to dynamically changing gas conditions. For example, a fluid containing red blood cells is flowed through the microfluidic device where the gas delivered to the gas channel contains about 20% oxygen, about 5% carbon dioxide and about 75% nitrogen. One or more measurements, for example a morphological measurement (e.g., cell sickling) or a kinetic measurement (e.g., cell velocity) can be made as the cells flow through the microfluidic device under high oxygen content. The gas delivered to the gas channel can then be switched to a gas having a low oxygen content (e.g., about 2% oxygen, about5% carbon dioxide and about 75% nitrogen) to regulate the oxygen content of the fluid containing red blood cells. One or more additional measurements may be taken over time to dynamically observe/measure one or more cell parameters in response to low oxygen conditions. For example, cell sickling time, or capillary obstruction ratio may be determined for a given cell sample when oxygen levels decrease. It should be appreciated that the device may be used to measure a cell-scale parameter in response to any gas or gas mixture and is not limited to the examples provided herein.

Devices containing one or more microfluidic channels can further contain a substantially planar transparent wall that defines a surface of at least one of the constrictions. This substantially planar transparent wall, which can be, for example, glass or plastic, permits observation into the microfluidic channel by microscopy so that at least one measurement of each cell that passes through one of the microfluidic channels can be obtained. In one example, the transparent wall has a thickness of 0.05 mm to 1 mm. In some cases, the transparent wall may be a microscope cover slip, or similar component. Microscope coverslips are widely available in several standard thicknesses that are identified by numbers, as follows: No. 0-0.085 to 0.13 mm thick, No. 1-0.13 to 0.16 mm thick, No. 1.5-0.16 to 0.19 mm thick, No. 2-0.19 to 0.23 mm thick, No. 3-0.25 to 0.35 mm thick, No. 4-0.43 to 0.64 mm thick, any one of which may be used as a transparent wall, depending on the device, microscope, cell size, and cell detection strategy.

In some embodiments, the transparent wall, or any wall of the microfluidic channel contains binding agents. Exemplary binding agents include antibodies, aptamers, or other suitable affinity capture reagents for binding to a target of interest, e.g., a cell.

The microfluidic channel(s) may have a height in a range of 0.5 μm to 100 μm, 0.1 μm to 100 μm, 1 μm to 50 μm, 1 μm to 50 μm, 10 μm to 40 μm, 5 μm to 15 μm, 0.1 μm to 5 μm, or 2 μm to 5 μm. The microfluidic channel(s) may have a height of up to 0.5 μm, 1 μm, 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, 10 μm, 20 μm, 30 μm, 40 μm, 50 μm, 75 μm, 100 μm, or more. In a specific embodiment, the microfluidic channel(s) have a height of 5.0 μm.

The microfluidic channel(s) may, in some cases, comprise 2, 3, 4, 5, 6, 7, 8, 9, 10, 15, 20, 25, 30, 35, 40, 45, 50, 75, 100, 200, or more constrictions, arranged in series. The microfluidic channel(s) may comprise 2 to 5, 2 to 10, 2 to 20, 2 to 50, 10 to 50, 10 to 100, or 50 to 200 constrictions, arranged in series, for example.

The microfluidic channel(s) may, in some cases, comprise 2, 3, 4, 5, 6, 7, 8, 9, 10, 15, 20, 25, 30, 35, 40, 45, 50, 75, 100, 200, or more constrictions, arranged in parallel. The microfluidic channel(s) may comprise 2 to 5, 2 to 10, 2 to 20, 2 to 50, 10 to 50, 10 to 100, or 50 to 200 constrictions, arranged in parallel, for example.

The device described above can further contain a reservoir fluidically connected with the one or more microfluidic channels, and a pump that perfuses fluid from the reservoir through the one or more microfluidic channels, and optionally, a microscope arranged to permit observation within the one or more microfluidic channels. The reservoir may contain cells suspended in a fluid. The fluidics connecting the reservoir to the microfluidic channel(s) may include one or more filters to prevent the passage of unwanted or undesirable components into the microfluidic channels.

The device may be designed and configured to create a pressure gradient from the channel inlet to the channel outlet of 0.05 Pa/μm, 0.1 Pa/μm, 0.15 Pa/μm, 0.2 Pa/μm, 0.25 Pa/μm, 0.3 Pa/μm, 0.35 Pa/μm, 0.4 Pa/μm, 0.45 Pa/μm, 0.5 Pa/μm, 0.55 Pa/μm, 0.6 Pa/μm, 0.65 Pa/μm, 0.7 Pa/μm, 0.75 Pa/μm, 0.8 Pa/μm, 0.85 Pa/μm, 0.9 Pa/μm, 0.95 Pa/μm, 1 Pa/μm, 2 Pa/μm, 3 Pa/μm, 4 Pa/μm, 5 Pa/μm, 10 Pa/μm, or more.

The device may be designed and configured to create a pressure gradient from the channel inlet to the channel outlet in a range of 0.05 Pa/μm to 0.1 Pa/μm, 0.1 Pa/μm to 0.3Pa/μm, 0.1 Pa/μm to 0.5 Pa/μm, 0.1 Pa/μm to 0.8 Pa/μm, 0.5 Pa/μm to 1 Pa/μm, 1 Pa/μm to 10 Pa/μm, for example. The pressure gradient may be linear or non-linear.

The device may be designed and configured to create a pressure (gauge pressure) in the channel of up to 50 Pa, 100 Pa, 200 Pa, 300 Pa, 400 Pa, 500 Pa, 600 Pa, 700 Pa, 800 Pa, 900 Pa, 1 kPa, 2 kPa, 5 kPa, 10 kPa or more. The device may be designed and configured to create a pressure (gauge pressure) in the channel in a range of 50 Pa to 200 Pa, 100 Pa to 500 Pa, 100 Pa to 800 Pa, 100 Pa to 1 kPa, 500 Pa to 5 kPa, or 500 Pa to 10 kPa.

The device may be designed and configured to create an average fluid velocity within the channel of up to 1 μm/s, 2 μm/s, 5 μm/s, 10 μm/s, 20 μm/s, 50 μm/s, 100 μm/s, or more. The device may be designed and configured to create an average fluid velocity within the channel in a range of 1 μm/s to 5 μm/s, 1 μm/s to 10 μm/s, 1 μm/s to 20 μm/s, 1 μm/s to 50 μm/s, 10 μm/s to 100 μm/s, or 10 μm/s to 200 μm/s, for example.

The device may be designed and configured to have a channel cross-sectional area, perpendicular to the flow direction, of 1 μm², 10 μm², 20 ium², 30 ium², 40 ium², 50 ium², 60 μm², 150 μm², 200 μm², 400 15 μm², 70 μm², 80 μm², 90 μm², 100 μm², 300 μm², 500 μm², 600 μm², 700 μm², 800 μm², 900 μm², 1000 μm², or more.

The device may be designed and configured to have a channel cross-sectional area, perpendicular to the flow direction, in a range of 1 μm² to 10 μm², 10 μm² to 50 μm², 50 μm² to 100 μm², 100 μm² to 500 μm², 500 μm² to 1500 μm², for example. The device may be designed and configured to produce any of a variety of different shear rates (e.g., up to 1000 s⁻¹). For example, the device may be designed and configured to produce a shear rate in a range of 10 s⁻¹ to 50 s⁻¹, 10 s⁻¹to 100 s⁻¹, 50 s⁻¹to 200 s⁻¹, 100 s⁻¹to 200 s⁻¹, 100 s⁻¹ to 500 s⁻¹, 50 s⁻¹ to 500 s⁻¹, or 50 s⁻or 1000 s⁻.

Alternatively or additionally, the device described herein further contains a heat transfer element, which can maintain the fluid at a predetermined temperature (e.g., a physiologically relevant temperature (e.g., a temperature that would be found in vivo in a healthy or diseased subject or one with a particular condition as provided herein), such as 30° C. to 45° C., preferably 37° C., 40° C. or 41° C.).

In some embodiments, non-microfluidic devices are provided. In some embodiments, the non-microfluidic device is AFM, optical tweezers, micropipette, magnetic twisting cytometer, cytoindenter, microindenter, nanoindenter, microplate stretcher, microfabricated post array detector, micropipette aspirator, substrate stretcher, shear flow detector, diffraction phase microscope, or tomographic phase microscope.

### Computational Methods, Systems and Devices

A computational framework is provided in some aspects that quantitatively predicts mechanical properties of cells. The computational framework uses as inputs, in some cases, information (e.g., transit characteristics) about the passage of a cell through the microfluidic devices disclosed herein. For example, a computational framework is provided in some aspects that quantitatively predicts mechanical properties of healthy and diseased red blood cells (RBCs) given the information about the passage of RBCs through micropores.

A computational approach for modeling cells by means of a Dissipative Particle Dynamics (DPD) model, or other appropriate model, provides a unique means to assess the influence of a variety of different properties on the deformation of a cell. Depending on the cell, the properties may include size, shape, membrane shear modulus, membrane viscosity, bending modulus, viscosity of internal fluid and suspending medium. In some aspects, each of these properties can be varied independently of each other in model simulations.

In some aspects, computational models provided herein have led to the development of numerical closed form functions that can predict mechanical properties of cells based on flow characteristics through a microfluidics device. Often the input parameters for the closed-form function include characteristics specific to the flow device used in the development of the model, and of the cell under investigation. For example, input parameters may include, dimensions of the constriction (micropore), applied pressure differential driving the flow, transit time of the object, and transit velocity of the object. The output of the closed-form function is typically a quantitative estimate of the value of a cell property, such as shear modulus or membrane viscosity. The approach can be generalized to constrictions of various dimensions, as disclosed herein, and any of the cells disclosed herein.

In some cases, methods are provided that involve performing one or more assays on one or more cells to obtain a measurement of one or more mechanical, physical or morphological properties; simulating, with at least one processor, flow of a fluid comprising more than one type of cell; and obtaining a closed-form equation with data from the simulation in combination with the measurement.

An illustrative example of the methods include at least obtaining data from at least one flow test performed on a fluid that contains more than one type of cell, and comparing the data with one or more predicted values calculated with at least one closed-form equation that correlates flow behavior to at least one material property (e.g., velocity, shear modulus, shear rate, shear stress, strain rate, yield stress, or hematocrit). Optionally, this method further includes one or more of: calculating the predicted values with the at least one closed-form equation, assessing the health of a subject from which the fluid is derived, and sorting and/or collecting one type of cell from another based on the comparison.

The flow test may be performed on a fluid under a predetermined set of microfluidic conditions, e.g., at a specific pressure, pressure gradient, velocity, etc. In one example, the flow test is performed by passing the fluid through one or more microfluidic channels, which can contain one or more constrictions or form part of a microfludic device (e.g., any of the microfludic devices described herein). In another example, the flow test is performed by passing the fluid through a microbead suspension, a flow cytometer, or a suspended microchannel resonator. A combination of different flow tests and/or mechanical or rheological assessments may be used in some cases.

The fluid can contain more than one type of cell (e.g., a mixture of both healthy and diseased cells), vesicles, biomolecular aggregates, platelet or particle, or a combination thereof. In one example, the fluid contains red blood cells, white blood cells, epithelial cells, or a mixture thereof. In another example, it contains cancer cells. In yet another example, the fluid (e.g., whole blood) contains T cells, B cells, platelets, reticulocytes, mature red blood cells, or a combination thereof. In some case, the fluid is substantially pure. The fluid may be whole-blood, serum, or plasma.

Any of the cells disclosed herein may be used in the methods. For example, epithelial cells of the cervix, pancreas, breast or bladder may be used. Red blood cells may be used, including, for example, fetal red blood cells, red blood cells infected with a parasite, red blood cells from a subject having or is suspected of having a disease, such as diabetes, HIV infection, anemia, cancer (e.g., a hematological cancer such as leukemia), multiple myeloma, monoclonal gammopathy of undetermined significance, or a disease that affects the spleen.

Flow test data can include a value for a transit characteristic, e.g., the velocity for one of the cells, the average velocity for a population of the cells, the distance traveled by one of the cells, the time for one of the cells to travel a certain distance, the average distance traveled by a population of the cells or the average time for a population of the cells to travel a certain distance.

A further illustrative method involves obtaining a value for one or more mechanical properties of a cell, determining a rheologic property (e.g., velocity) of the fluid described herein comprising the cell using a closed-form equation that correlates the mechanical property with the rheologic property, and optionally, making a prediction about the health of a subject (e.g., a subject having sickle cell disease, malaria or diabetes) based on the determination of the rheologic property. The one or more mechanical properties can be measured by, e.g., AFM, optical tweezers, micropipette, magnetic twisting cytometer, cytoindenter, microindenter, nanoindenter, microplate stretcher, microfabricated post array detector, micropipette aspirator, substrate stretcher, shear flow detector, diffraction phase microscope, or tomographic phase microscope. The prediction can include an assessment of the aggregation of the cells in the fluid.

Data comparison can be performed using at least one processor. The at least one close-form equation employed in this step can be developed from one or more simulations of flow of a fluid in combination with experimental data. The one or more stimulations can be performed using dissipative particle dynamics model, a stochastic bond formation/dissociation model, or other appropriate model. The experimental data preferably is from an assay that measures membrane shear modulus, membrane bending rigidity, membrane viscosity, interior/exterior fluid viscosities, or a combination thereof, on a cell. However, any of a variety of experimental inputs may be used.

The step of assessing the health of a subject from which a fluid or cell is derived can be performed by determining the presence or absence of a disease or condition in the subject or determining the stage of a disease or condition.

An further illustrative example of the methods include obtaining data for one or more mechanical properties of a cell, and determining one or more predicted values of flow behavior. The one or more predicted values are determined with at least one closed-form equation that correlates flow behavior of any of the fluids or cells described herein to the one or more material properties (e.g., mechanical and/or rheological properties) of the fluid or a component thereof. For example, one or more predicted values may be determined with at least one closed-form equation that correlates flow behavior of blood to the one or more rheological properties of the blood. Information regarding the rheological properties of the blood may be used to evaluate the likelihood of a clinical condition, e.g., aggregate formation, capillary occlusion in the brain, heart or other tissue, etc. in a subject. Thus, the closed form equation together with information regarding the flow behavior of a biological fluid obtained from a subject may be used in some case to diagnosis or evaluate a disease or condition in the subject.

Apparatus are provided in some aspects for performing at least one of the methods described herein. An illustrative example of such an apparatus contains a device for performing a flow test on a fluid, a computer system for obtaining data from the flow test and comparing the data with one or more predicted values. Alternatively, the apparatus contains a device for obtaining data for one or more mechanical properties of a cell, and a computer system for obtaining the data and determining one or more predicted values. The predicted value(s) can be calculated with at least one closed-form equation that correlates flow behavior of the cell-containing fluid described herein to the one or more mechanical properties.

Also provided are methods for manufacturing a diagnostic test apparatus that contains a device either for performing a flow test or for determining one or more mechanical properties of a cell; and a computing device that predicts at least one rheologic property of a sample (e.g., any of the cell-containing fluids described herein) based on flow behavior measured on the sample passing through the device, compares a value for a measurement of a sample as it passes through the device, or calculates one or more predicted values for flow behavior of the fluid described herein. Further methods may include generating, with at least one processor and a model of cells within a fluid, a closed-form equation relating at least one parameter of flow of the fluid through the device to the at least one rheologic property; and encoding the closed-form equation in software configured for execution on the computing device. In another example, this method includes comparing, with at least one processor, the value with one or more predicted values calculated with a closed-form equation relating at least one parameter of flow of the fluid to at least one rheologic property; and encoding the one or more predicted values in software configured for execution on the computing device.

In some embodiments, the apparatus comprises a non-microfluidic device. In some embodiments, the non-microfluidic device is AFM, optical tweezers, micropipette, magnetic twisting cytometer, cytoindenter, microindenter, nanoindenter, microplate stretcher, microfabricated post array detector, micropipette aspirator, substrate stretcher, shear flow detector, diffraction phase microscope, or tomographic phase microscope.

Manufacturing methods include calculating, with at least one processor, one or more predicted values with the one or more mechanical properties, the one or more predicted values being calculated with a closed-form equation relating at least one parameter of flow of the fluid to the one or more mechanical properties; and encoding the one or more predicted values in software configured for execution on the computing device.

In addition, the present invention features a method including an inputting step and a calculating or comparing step. The inputting step can be performed by inputting a value for a measurement of any of the cell-containing fluids described herein as it passes through a flow test device. Alternatively, it is performed by inputting a value for one or more mechanical properties of a cell. The calculating step can be performed by calculating at least one mechanical or rheological property with a closed-form equation and the inputted value, the equation relating at least one parameter of flow of the fluid through the device to the at least one mechanical or rheological property, or by calculating one or more predicted values for flow behavior of any of the fluids described herein, the one or more predicted values being calculated with a closed-form equation relating at least one parameter of flow of the fluid the one or more mechanical properties. The comparing step may involve comparing the value with a predicted value from a calculation with at least one closed-form equation that correlates flow behavior to at least one mechanical or rheological property. Any of the methods described in this paragraph can further involve calculating the predicted value with the closed-form equation.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the invention may be embodied as a computer readable medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other non-transitory, tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above. As used herein, the term “non-transitory computer-readable storage medium” encompasses only a computer-readable medium that can be considered to be a manufacture (i.e., article of manufacture) or a machine.

The terms “program” or “software” are used herein in a generic sense to refer to any type of computer code or set of computer-executable instructions that can be employed to program a computer or other processor to implement various aspects of the present invention as discussed above. Additionally, it should be appreciated that according to one aspect of this embodiment, one or more computer programs that when executed perform methods of the present invention need not reside on a single computer or processor, but may be distributed in a modular fashion amongst a number of different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Various aspects of the present invention may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

### Methods of Measuring Cell Properties Under Controlled Gas Conditions

Methods are provided herein for evaluating, characterizing, and/or assessing mechanical, morphological, kinetic, rheological or hematological properties of cells under controlled gas conditions. In particular, methods are provided for measuring, evaluating and/or characterizing dynamic mechanical responses of biological cells, e.g., red blood cells, white blood cells, reticulocytes, platelets, etc. The methods typically involve obtaining measurements of cell deformability, cell velocity and cell shape. Measurements of cell deformability often involve an assessment of the transit time of one or more cells through one or more constrictions within a fluid channel of a microfluidic device, or an assessment of another parameter indicative of a resistance to deformation. In some cases, the methods may be carried out in a high throughput manner. In some aspects, methods are provided that are useful for diagnosing, assessing, characterizing, evaluating, and/or predicting disease based on transit characteristics of cells, e.g., red blood cells, platelets, cancer cells, and tissues, e.g., blood in microfluidic devices. In further aspects, methods are provided that are useful for measuring changes in cell properties or characteristics in response to changes in the concentration of one or more gasses. As one example, the transit characteristics of a red blood cell through one or more constrictions of a microfluidic device are measured at high oxygen content (e.g., 20% oxygen) and low oxygen content (e.g., 2% oxygen).

Some aspects of the disclosure relate to determining cell properties in response to repetitive or cyclical changes in the concentration of one or more gases (e.g., alternating between relatively high and low concentrations of a gas in a fluid). In some aspects, methods are provided that are useful for measuring changes in cell properties or characteristics in response to one or more cycles of a gas concentration. For example, in some embodiments one or more changes in cell properties or characteristics are measured in response to one or more cycles of an oxygen, a nitrogen, a carbon dioxide, a carbon monoxide, a nitric oxide, a nitrous oxide, a nitrogen dioxide, or a methane gas concentration. However, it should be appreciated that cell properties or characteristics may be determined in response to one or more cycles of any suitable gas concentration. In some embodiments, one or more changes in cell properties or characteristics are measured in response to one or more changes in oxygen concentration.

A cycle of a gas concentration refers to a change from a relatively high gas concentration (e.g., 20% oxygen) to a relatively low gas concentration (e.g., 2% oxygen) and back to a relatively high gas concentration. A cycle of a gas concentration also refers to a change from a relatively low gas concentration (e.g., 2% oxygen) to a relatively high gas concentration (e.g., 20% oxygen) and back to a relatively low gas concentration. In some embodiments a cycle of a gas concentration refers to a change from a relatively high oxygen concentration to a relatively low oxygen concentration and back to a relatively high oxygen concentration, referred to herein as a deoxygenation (DeOxy) cycle. In some embodiments a cycle of a gas concentration refers to a change from a relatively low oxygen concentration to a relatively high oxygen concentration and back to a relatively low oxygen concentration, referred to herein as a reoxygenation (ReOxy) cycle. In some embodiments a change from a relatively high gas concentration (e.g., of oxygen) to a relatively low gas concentration refers to a decrease in gas concentration of at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or at least 100% in a gas or fluid. In some embodiments a change from a relatively low gas concentration (e.g., of oxygen) to a relatively high gas concentration refers to an increase in gas concentration of at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, or 100% in a gas or fluid.

Some aspects of the disclosure relate to determining cell properties in response to one or more cycles of a gas concentration. In some embodiments one or more cell properties are determined after being exposed to at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 600, 700, 800, 900, or at least 1000 cycles of a gas concentration. In some embodiments, one or more cell properties are determined after being exposed to at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 600, 700, 800, 900, or at least 1000 deoxygenation (DeOxy) cycles. In some embodiments, one or more cell properties are determined after being exposed to at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 350, 400, 450, 500, 600, 700, 800, 900, or at least 1000 reoxygenation (ReOxy) cycles.

In some embodiments, the cycles of a gas concentration provided herein may be performed for any suitable duration of time, which may depend on, among other factors, the intended purpose or the nature of the cells (e.g., healthy or diseased cells). In some embodiments, the duration of two or more consecutive cycles are the same. For example, in some embodiments, two or more consecutive cycles may be 360 seconds long. In some embodiments, the length of two or more consecutive cycles are different. For example a first cycle may be 360 seconds long and a second cycle may be 400 seconds long. In some embodiments, the length of two or more consecutive cycles may be increased. In some embodiments, the length of two or more consecutive cycles may be decreased. In some embodiments a cycle is from 5 seconds (5 s) to 1 hour (1 h) long. However, it should be appreciated that a cycle may be any suitable duration and any exemplary cycle durations provided herein are not intended to be limiting. In some embodiments, a cycle is from 5 s to 20 s, from 5 s to 100 s, from 5 s to 200 s, from 5 s to 400 s, from 5 s to 600 s, from 5 s to 1000s, from 5 s to 20 min, from 5 s to 30 min, from 5 s to 40 min, from 5 s to 50 min, from 100 s to 200s, from 100 s to 400 s, from 100 s to 600 s, from 100 s to 1000 s, from 100 s to 20 min, from 100 s to 30 min, from 100 s to 40 min, from 100 s to 50 min from 100 s to 1 h, from 200 s to 400 s, from 200 s to 600 s, from 2 s to 1000 s, from 200 s to 20 min, from 200 s to 30 min, from 200 s to 40 min, from 200 s to 50 min from 200 s to 1 h, from 400 s to 600 s, from 400 s to 1000 s, from 400 s to 20 min, from 400 s to 30 min, from 400 s to 40 min, from 400 s to 50 min, or from 400 s to 1 h in duration.

In some embodiments, the duration of time that a gas is at a relatively high concentration, within a cycle, may vary. In some embodiments, the duration of time that a gas is at a relatively low concentration, within a cycle, may vary. In some embodiments, the duration of time at which a gas is at a relatively high concentration and the duration of time at which a gas is at a relatively low concentration, within a cycle, is the same. In some embodiments, the duration of time at which a gas is at a relatively high concentration and the duration of time at which a gas is at a relatively low concentration, within a cycle, is different. In some embodiments, the duration of time at which a gas is at a relatively high concentration is greater than the duration of time at which a gas is at a relatively low concentration, within a cycle. In some embodiments, the duration of time at which a gas is at a relatively high concentration is at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 100%, 500%, 1000%, or at least 5000% greater than the duration of time at which a gas is at a relatively low concentration, within a cycle. In some embodiments, the duration of time at which a gas is at a relatively high concentration, within a cycle, is from 1 s to 20 s, from 1 s to 100 s, from 1s to 200 s, from 1 s to 400 s, from 1 s to 600 s, from 1 s to 1000s, from 1 s to 20 min, from 1 s to 30 min, from 1 s to 40 min, from 1 s to 50 min, from 1 s to 1 h, from 100 s to 200 s, from 100 s to 400 s, from 100 s to 600 s, from 100 s to 1000 s, from 100 s to 20 min, from 100 s to 30 min, from 100 s to 40 min, from 100 s to 50 min from 100 s to 1 h, from 200 s to 400 s, from 200 s to 600 s, from 2 s to 1000 s, from 200 s to 20 min, from 200 s to 30 min, from 200 s to 40 min, from 200 s to 50 min from 200 s to 1 h, from 400 s to 600 s, from 400 s to 1000s, from 400 s to 20 min, from 400 s to 30 min, from 400 s to 40 min, from 400 s to 50 min, or from 400 s to 1 h in duration. In some embodiments, the duration of time at which a gas is at a relatively high concentration is less than the duration of time at which a gas is at a relatively low concentration, within a cycle. In some embodiments, the duration of time at which a gas is at a relatively high concentration is at least 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 100%, 500%, 1000%, or at least 5000% less than the duration of time at which a gas is at a relatively low concentration, within a cycle. In some embodiments, the duration of time at which a gas is at a relatively low concentration, within a cycle, is from 1 s to 20 s, from 1 s to 100 s, from 1 s to 200 s, from 1 s to 400 s, from 1 s to 600 s, from 1 s to 1000s, from 1 s to 20 min, from 1 s to 30 min, from 1 s to 40 min, from 1 s to 50 min, from 1 s to 1 h, from 100 s to 200 s, from 100 s to 400 s, from 100 s to 600 s, from 100 s to 1000 s, from 100 s to 20 min, from 100 s to 30 min, from 100 s to 40 min, from 100 s to 50 min from 100 s to 1 h, from 200 s to 400 s, from 200 s to 600 s, from 2 s to 1000 s, from 200 s to 20 min, from 200 s to 30 min, from 200 s to 40 min, from 200 s to 50 min from 200 s to 1 h, from 400 s to 600 s, from 400 s to 1000s, from 400 s to 20 min, from 400 s to 30 min, from 400 s to 40 min, from 400 s to 50 min, or from 400 s to 1 h in duration.

In some cases, the methods involve acquiring microscopic measurements, e.g., fluorescence measurements, on cells passing through one or more constrictions of a microfluidic device at a controlled gas concentration. A combination of acquired microfluidic data (e.g., gas concentration, flow, pressure, transit time, constriction geometry, flow length, etc.) and microscopic data (e.g., morphology and/or the presence or absence of a cell surface markers), enables a population-based correlation between cellular and/or biochemical properties and dynamic mechanical properties, such as deformability.

### Characterizing Cells

Methods for characterizing deformability of one or more cells are provided herein. The methods typically involve perfusing a fluid containing one or more deformable objects through a microfluidic channel that includes at least one constriction and determining a transit characteristic of the one or more deformable objects at a controlled gas concentration. For example, at an oxygen concentration of 20%. The transit characteristic may be, for example, the transit time for the one or more cells to travel from a first position within the microfluidic channel that is upstream of a constriction to a second position within the microfluidic channel that is downstream of a constriction. The transit characteristic may be, for example, the average velocity of the one or more deformable objects between a first position within the microfluidic channel that is upstream of a constriction and a second position within the microfluidic channel that is downstream of a constriction.

The transit characteristic may be measured as a function of time and/or as a function of gas concentration. In some embodiments, a transit characteristic of one or more cells is measured at one or more gas concentrations. For example a transit characteristic of one or more red blood cells from a subject is measured at a low oxygen concentration (e.g., 2% oxygen). Upon increase of the oxygen concentration (e.g., 20% oxygen), another transient characteristic of one or more cells can be measured. It should be appreciated that the methods, provided herein, allow for the real-time observation of hypoxia-induced changes in transient characteristics. The measurements of transient characteristics of the cells, described herein, may be used to determine the fraction of obstructed cells.

It should be appreciated that methods provided herein allow for the real-time observation of hypoxia-induced changes in cell morphology. For example, cell sickling in response to low oxygen concentrations. The methods provided also allow for the real-time observation of reoxygenation induced cell shape recovery. For example the transition of sickled cells into normal disk shaped red blood cells. In other embodiments, a morphological characteristic, such as a cell shape change (e.g., sickling, a sphericity change, and aspect ratio change or a texture change), of one or more cells is measured at one or more gas concentrations. For example a morphological characteristic of one or more red blood cells from a subject is measured at a low oxygen concentration (e.g., 2% oxygen). Upon increase of the oxygen concentration (e.g., 20% oxygen), another transient characteristic of one or more cells can be measured. The measurements of cell morphology of the cells may be used to determine the fraction of abnormally shaped (e.g., sickled) cells. It should be appreciated that methods provided herein allow for the real-time observation of hypoxia-induced changes in cell morphology. For example, cell sickling in response to low oxygen concentrations.

The methods provided also allow for the real-time observation of reoxygenation induced cell shape recovery. For example the transition of sickled cells, cells with a rough texture, or spiky cells into normal disk shaped red blood cells. Accordingly, the methods, described herein, allow for the simultaneous measurement of cell shape changes over time and cell transit characteristics in response to changes in gas concentration, for example, cell sickling delay time and sickled fraction can be simultaneously measured in real-time in response to decreased oxygen concentration.

The methods, described herein, may be used to determine the fraction of obstructed cells, the fraction of cells with an abnormal shape and/or texture, the capillary obstruction ratio, the delay time of an abnormal cell shape change, and/or the delay time of recovering from an abnormal cell shape change.

The transit characteristics may be determined in any of a variety of ways. Typically, the transit characteristic determination involves performing microscopy to acquire photomicrographic images of the cell as it passes through the channel. The object can be tracked manually, e.g., by examining the images by eye, or automatically, by implementing an image processing and/or image object tracking algorithm. For example, the transit characteristic may be determined by acquiring a first photomicrographic image of the one or more cells at the first position and acquiring a second photomicrographic image of the one or more deformable objects at the second position, and determining the duration between acquisition of the first photomicrographic image and acquisition the second photomicrographic image. The duration, in this example, is the transit time. The average velocity can be readily determined, in this example, by computing the ratio of the transit time to the transit distance. The transit characteristics or changes in transit characteristics, may be determined over time in response to changes in gas concentration.

The constriction typically has an inlet orifice, outlet orifice and/or conduit that has a geometry that causes the object to deform as it passes through the constriction. Thus, the size and/or shape of the constriction may be configured so as to require that the cell deform in order to pass through the constriction. For example, the constriction may have an inlet orifice, outlet orifice, and/or conduit having a dimension (e.g., diameter), perpendicular to the flow path, that is smaller in length than the diameter of the object, such that the object must deform in order to pass through the constriction.

In some cases, the methods involve perfusing a fluid containing one or more cells (e.g., blood cells) through a microfluidic channel that includes a plurality of constrictions arranged in series. The plurality of constrictions are typically arranged in series such that a flow path through each constriction of the plurality is longitudinally aligned with a flow path through each other constriction of the plurality. In this configuration, the one or more cells can be tracked, e.g., by microscopy, as it enters or passes through each constriction of the plurality. However, the methods and devices are not so limited and configurations are envisioned where the plurality of constrictions are arranged sequentially such that a flow path through each constriction of the plurality is not longitudinally aligned with a flow path through each other constriction of the plurality.

The deformability of a cell may be characterized, in some cases, by evaluating the effects of constriction geometries on the transit of a cell through a microfluidic channel. For example, the transit times of a cell through two or more different constrictions (e.g., constrictions having different geometries, e.g., different inlet orifice, outlet orifice, and/or conduit geometries) may be used to define a signature that characterizes the deformability of the cell.

### Diagnostic Methods

Also disclosed herein are methods for detecting a condition or disease in a subject. “Subject,” as used herein, refers to any animal. Typically a subject is a mammal, particularly a domesticated mammal (e.g., dogs, cats, etc.), primate, human or laboratory animal. In certain embodiments, the subject is a human. In certain embodiments, the subject is a laboratory animal such as a mouse or rat. A subject under the care of a physician or other health care provider may be referred to as a “patient.” In the context of diagnosis, typically the subject has or is suspected of having a disease. The diagnostic methods disclosed herein may be used in combination with one or more known diagnostic approaches in order to diagnose a subject as having a disease.

The methods typically involve obtaining a biological sample from the subject. As used herein, the phrase “obtaining a biological sample” refers to any process for directly or indirectly acquiring a biological sample from a subject. For example, a biological sample may be obtained (e.g., at a point-of-care facility, e.g., a physician's office, a hospital, laboratory facility) by procuring a tissue or fluid sample (e.g., blood draw, marrow sample, spinal tap) from a subject. Alternatively, a biological sample may be obtained by receiving the biological sample (e.g., at a laboratory facility) from one or more persons who procured the sample directly from the subject. The biological sample may be, for example, a tissue (e.g., blood), cell (e.g., hematopoietic cell such as hematopoietic stem cell, leukocyte, or reticulocyte, stem cell, or plasma cell), vesicle, biomolecular aggregate or platelet from the subject.

The biological sample typically serves as a test agent for a deformability assay where the level of a gas is regulated. The results of the deformability assay of the test agent are often indicative of the disease status of the subject. For example, in some cases, deformability of the test agent, e.g., a blood cell, at a given gas concentration, such as a hypoxic gas concentration (e.g., 2% oxygen), is indicative of the presence of the condition or disease in the subject. In some cases, the deformability assay involves perfusing a fluid, at a regulated gas concentration, containing a test agent through a microfluidic channel that comprises a constriction, such that the test agent passes through the constriction, and deforms as it passes through the constriction. The assay further involves determining a transit characteristic of the test agent as it moves through the microfluidic channel and comparing the transit characteristic to an appropriate standard. The results of the comparison are typically indicative of whether the subject has the condition or disease. Thus, the subject may be diagnosed as having the condition or disease based on the results of the deformability assay, in some cases. In some embodiments, a method for analyzing, diagnosing, detecting, or determining the severity of a condition or disease in a subject, includes (a) perfusing a fluid comprising one or more cells from the subject through the any of the microfluidic devices, described herein, where the level of one or more gases is regulated, (b) determining a property of one or more of the cells; and (c) comparing the property to an appropriate standard, wherein the results of the comparison are indicative of the status of the condition or disease in the subject.

Any appropriate condition or disease of a subject may be evaluated using the methods herein, typically provided that a test agent may be obtained from the subject that has a material property (e.g., deformability, shear modulus, viscosity, Young's modulus, etc.) that is indicative of the condition or disease. The condition or disease to be detected may be, for example, a fetal cell condition, HPV infection, or a hematological disorder, such as sickle cell disease, sickle cell trait (SCT), spherocytosis, ovalocytosis, alpha thalassemia, beta thalassemia, delta thalassemia, malaria, anemia, diabetes, leukemia, hematological cancer, infectious mononucleosis, HIV, malaria, leishmaniasis, babesiosis, monoclonal gammopathy of undetermined significance or multiple myeloma. Examples of hematological cancer include, but are not limited to, Hodgkin's disease, Non-Hodgkin's lymphoma, Burkitt's lymphoma, anaplastic large cell lymphoma, splenic marginal zone lymphoma, hepatosplenic T-cell lymphoma, angioimmunoblastic T-cell lymphoma (AILT), multiple myeloma, Waldenstrm macroglobulinemia, plasmacytoma, acute lymphocytic leukemia (ALL), chronic lymphocytic leukemia (CLL), B cell CLL, acute myelogenous leukemia (AML), chronic myelogenous leukemia (CML), T-cell prolymphocytic leukemia (T-PLL), B-cell prolymphocytic leukemia (B-PLL), chronic neutrophilic leukemia (CNL), hairy cell leukemia (HCL), T-cell large granular lymphocyte leukemia (T-LGL) and aggressive NK-cell leukemia. The foregoing diseases or conditions are not intended to be limiting. It should thus be appreciated that other appropriate diseases or conditions may be evaluated using the methods disclosed herein.

Methods are also provided for detecting and characterizing a leukocyte-mediated condition or disease in a controlled gas environment. For example, methods are provided for detecting and characterizing a leukocyte-mediated condition or disease associated with the lungs of a subject being highly susceptible to injury, possibly due to activated leukocytes with altered deformability, having altered ability to circulate through the pulmonary capillary bed. Methods such as these, and others disclosed herein, can also be applied to detect and/or characterize septic shock (sepsis) that is associated with both rigid and activated neutrophils. Such neutrophils can, in some cases, occlude capillaries and damage organs where changes in neutrophil cytoskeleton are induced by molecular signals leading to decreased deformability.

Further, certain methods of the invention provide for measurement of cytoadhesive properties of a cell population, in combination with or separate from measurement of the deformability of the cell population. The combination of determining cytoadhesive properties and the deformative properties of a cell population, particularly a cell population containing a plurality of different cell types (e.g., red blood cells and white blood cells), may be used to generate a “Health Signature” that comprises an array of properties that can be tracked in a subject over a period of time. Such a Health Signature may facilitate effective monitoring of a subject's health over time. Such monitoring may lead to an early detection of potential acute or chronic infection, or other disease, disorder, fitness, or condition. In some cases, further, knowledge of the overall rheology of a material, along with either the deformative or cytoadhesive property of a cell, allows the determination of the other property.

A method for detecting a condition or disease (e.g., sickle cell disease) in a subject may, in some cases, include at least the following steps: (a) obtaining blood sample from the subject, the sample containing a red blood cell (b) analyzing a mechanical property of the blood sample at a regulated gas level using a device; and (c) comparing the mechanical property to an appropriate standard. The results of the comparison are typically indicative of the status of the condition or disease in the subject. In one example, the device is a microfluidic channel with a gas permeable membrane or film. In another example, the device is a microfluidic channel with a gas permeable membrane or film and a gas channel. The deformable object, in this example, typically has a mechanical property, the value of which is indicative of the presence of sickle cell disease. In one example, the method is used to determine the severity of the disease based on differences in mechanical properties. In another example, the method is used to predict the likelihood that a subject will undergo vaso-occlusion crisis based on differences in mechanical properties. In such methods, the methods may be performed under different regulated gas conditions

An “appropriate standard” is a parameter, value or level indicative of a known outcome, status or result (e.g., a known disease or condition status). An appropriate standard can be determined (e.g., determined in parallel with a test measurement) or can be pre-existing (e.g., a historical value, etc.). The parameter, value or level may be, for example, a transit characteristic (e.g., transit time), a value representative of a mechanical property, a value representative of a rheological property, etc. For example, an appropriate standard may be the transit characteristic of a test agent obtained from a subject known to have a disease, or a subject identified as being disease-free. In the former case, a lack of a difference between the transit characteristic and the appropriate standard may be indicative of a subject having a disease or condition. Whereas in the latter case, the presence of a difference between the transit characteristic and the appropriate standard may be indicative of a subject having a disease or condition. The appropriate standard can be a mechanical property or rheological property of a cell obtained from a subject who is identified as not having the condition or disease or can be a mechanical property or rheological property of a cell obtained from a subject who is identified as having the condition or disease.

The magnitude of a difference between a parameter, level or value and an appropriate standard that is indicative of known outcome, status or result may vary. For example, a significant difference that indicates a known outcome, status or result may be detected when the level of a parameter, level or value is at least 1%, at least 5%, at least 10%, at least 25%, at least 50%, at least 100%, at least 250%, at least 500%, or at least 1000% higher, or lower, than the appropriate standard. Similarly, a significant difference may be detected when a parameter, level or value is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than the level of the appropriate standard. Significant differences may be identified by using an appropriate statistical test. Tests for statistical significance are well known in the art and are exemplified in Applied Statistics for Engineers and Scientists by Petruccelli, Chen and Nandram Reprint Ed. Prentice Hall (1999).

### Monitoring Efficacy of Therapeutic Agents and Testing Candidate Therapeutic Agents

Methods are also provided for testing candidate therapeutic agents for treating a condition or disease in a subject. The methods typically involve: (a) obtaining a biological sample from a subject comprising a cell; (b) perfusing a fluid comprising one or more cells from the subject through any of devices, described herein, where the level of a gas is regulated; (c) determining a property of one or more of the cells; (d) contacting the biological sample comprising a cell with the therapeutic; (e) perfusing a fluid comprising the product of (d) through any of devices, described herein, where the level of a gas is regulated; (f) determining a property of one or more of the cells from (e); and (g) comparing the property of one or more cells from (c) with the property of one or more cells from (f), wherein the results of the comparison are indicative of the effectiveness of the therapeutic. Methods are also provided for monitoring the efficacy of a therapeutic in a subject. The methods typically involve: (a) perfusing a fluid comprising one or more cells from the subject through any of devices, described herein, where the level of a gas is regulated; (b) determining a property of one or more of the cells; (c) treating the subject with the therapeutic agent; and (d) repeating steps (a) and (b) at least once wherein a difference in the property of one or more cells is indicative of the effectiveness of the therapeutic agent.

In some embodiments, the appropriate standard is the value of a transit characteristic for a test agent at a regulated gas concentration that has been contacted with a control therapeutic agent (e.g., hydroxyurea or 5-hydroxymethylfurfural). Typically, a control therapeutic agent is a molecule that has a known effect on deformability of a test agent and that is effective for treating the condition or disease. Thus, comparing the transit characteristic of a candidate therapeutic agent with that of a control therapeutic agent provides a basis for identifying candidate therapeutic agents that are likely to be useful for treating the disease or condition. For example, a candidate therapeutic agent that results in the same or a similar value for a particular transit characteristic as that of a control therapeutic agent that is known to be effective for treating the disease or condition is likely to be an agent that is also effective for treating the disease or condition.

By example, this method may be used to identify candidate therapeutic agents that improve blood flow in subjects with circulation problems such as sickle cell disease, leg ulcers, pain from diabetic neuropathy, eye and ear disorders, and altitude sickness. Similarly for subjects with aggregation or clotting disorders of cells or insufficient delivery of essential chemicals such as oxygen to the brain in subjects with strokes from blood clots.

Typically the therapeutic agent or candidate therapeutic agent is a small molecule or pharmaceutical agent. “Small molecule” refers to organic compounds, whether naturally-occurring or artificially created (e.g., via chemical synthesis) that have relatively low molecular weight and that are not proteins, polypeptides, or nucleic acids. Small molecules are typically not polymers with repeating units. In certain embodiments, a small molecule has a molecular weight of less than about 1500 g/mol. In certain embodiments, the molecular weight of the polymer is less than about 1000 g/mol. Also, small molecules typically have multiple carbon-carbon bonds and may have multiple stereocenters and functional groups.

“Pharmaceutical agent,” also referred to as a “drug,” is used herein to refer to an agent that is administered to a subject to treat a disease, disorder, or other clinically recognized condition that is harmful to the subject, or for prophylactic purposes, and has a clinically significant effect on the body to treat or prevent the disease, disorder, or condition. Therapeutic agents include, without limitation, agents listed in the United States Pharmacopeia (USP), Goodman and Gilman's The Pharmacological Basis of Therapeutics, 10^{th} Ed., McGraw Hill, 2001; Katzung, B. (ed.) Basic and Clinical Pharmacology, McGraw-Hill/Appleton & Lange; 8th edition (September 21, 2000); Physician's Desk Reference (Thomson Publishing), and/or The Merck Manual of Diagnosis and Therapy, 17^{th} ed. (1999), or the 18^{th} ed (2006) following its publication, Mark H. Beers and Robert Berkow (eds.), Merck Publishing Group, or, in the case of animals, The Merck Veterinary Manual, 9^{th} ed., Kahn, C. A. (ed.), Merck Publishing Group , 2005.

### EXAMPLES

In order that the invention described herein may be more fully understood, the following examples are set forth. The examples described in this application are offered to illustrate the devices, compounds, pharmaceutical compositions, and methods provided herein and are not to be construed in any way as limiting their scope.

### Example 1

### Kinetics of Sickle Cell Biorheology and Implications for Vaso-Occlusive Crisis

### Microfluidic Platform.

An in vitro model with a well-defined vascular structure and a well-controlled hypoxia condition, would serve as an ideal tool to investigate many complex pathophysiological processes in vaso-occlusion. Several methods have been developed to mimic oxygen depletion whereby HbS polymerization and subsequent cell sickling can be triggered; they include: long-term gas perfusion at low pO₂ level (13, 27), DeOxy-medium exchange (25, 28), reducing agents (29-31), and laser photo-dissociation of carbon monoxide (22, 32). Along with complex in vivo models that reflect the dynamic response of cells, an in vitro model would have the potential to predict the conditions that would lead to vaso-occlusion and to improve the assessment of disease severity by quantifying the individual parameters that modulate vaso-occlusion.

A microfluidic platform (FIG. 1A-B) that mimics the rheology of microcirculation in vivo was designed. It has been used to characterize the isolated effects of cell morphologic sickling, unsickling, and altered cell rheology. With this design, the possible correlations of these effects to hematological parameters (e.g. % HbS), cell density, and hydroxyurea (HU) therapy were determined in a systematic and controlled manner,.

Cell sickling was measured using a double-layer device with a cell channel (5 μm high), a gas channel (100 μm high) and an in-between PDMS film 150 μm in thickness (FIG. 1A). The O₂ concentration was controlled by exchanging gas flow in the channel through the PDMS membrane that is gas-permeable (33). While it is known (34) that the morphology of sickled cells depends on the DeOxy rate, heterogeneity was observed in cell morphology at the same DeOxy rate. Sickle RBCs typically form spiky edges and dark coarse texture due to intracellular HbS polymerization, the visual identification of which was enhanced by a band-pass filter (FIG. 1B). Thus the sickled cells were defined as those obviously distorted from their original shape and texture under Oxy state (O₂ concentration 20%) to the DeOxy state (O₂ concentration <5%). This visual determination of cell sickling was further confirmed with an independent single cell rheology test, where similar trends were observed in cell sickling and single-cell capillary obstruction. The kinetics of cell sickling was quantified by two parameters, sickled fraction (fraction of all RBCs in the sample that are sickled) and the delay time of cell sickling (the time elapsed between the initiation of DeOxy and the point when a cell shows optically visible features of morphologic sickling). The delay time of cell unsickling was defined as the time elapsed between the initiation of reoxygenation (ReOxy) and the point when the RBC fully recovered its pre-sickle morphology in a visibly identifiable manner.

Individual cell rheology was measured using a microfluidic channel that consisted of periodic obstacles, forming micro-gates 4 μm wide, 5 μm deep and 15 μm long (FIG. 1C). Cell velocity was measured as average velocity of individual RBCs flowing through periodic gates under a constant differential pressure. Obstruction fraction was determined as the ratio of obstructed RBCs to all RBCs entering into the micro-gate arrays during the DeOxy period.

### Kinetics of Cell Sickling and Unsickling.

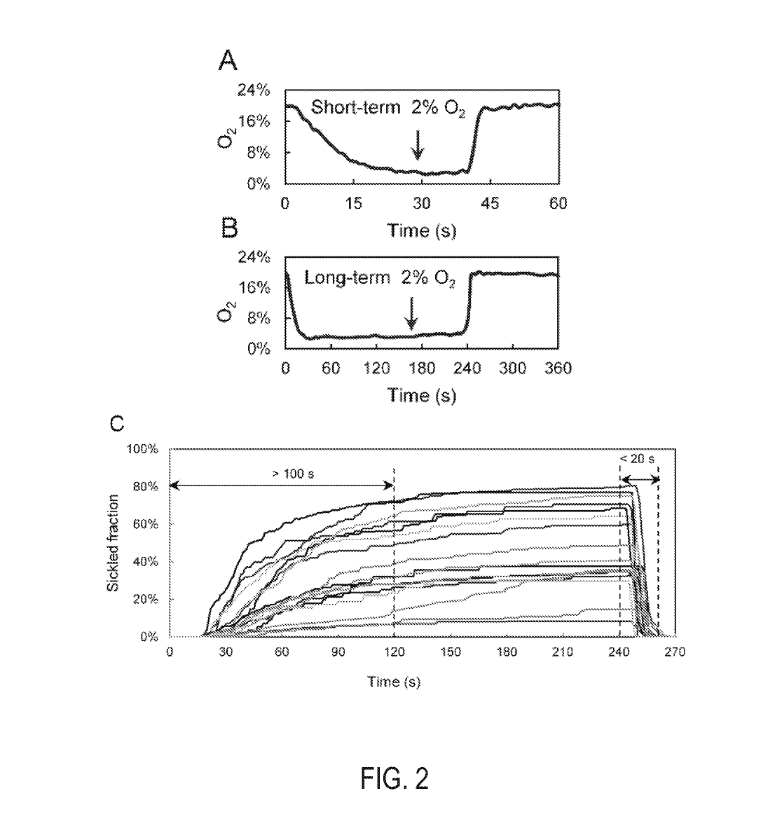

Kinetics of individual cell sickling and unsickling under transient hypoxia conditions was quantified by the delay time and the maximum sickled fraction on 25 SCD patient samples: 7 patients without HU therapy (off-HU) and 18 patients with HU therapy (on-HU) (see patient's HU status in Materials and Methods Text). Short-term and a long-term hypoxia conditions were created to simulate normal and retarded transit scenarios in microvasculature (FIGS. 2A-B). Representative cell sickling profiles upon changes in O₂ concentration are shown (FIG. 2C). Compared to the relatively long sickling process (>100 s for sickled fraction rising from zero to a saturated, maximum level), the unsickling process after ReOxy was much faster (<20 s for sickled fraction reducing from the saturated level to zero), disregarding the small discrepancy in the DeOxy and ReOxy rates (<20 s). This observation applied to all patient sample.

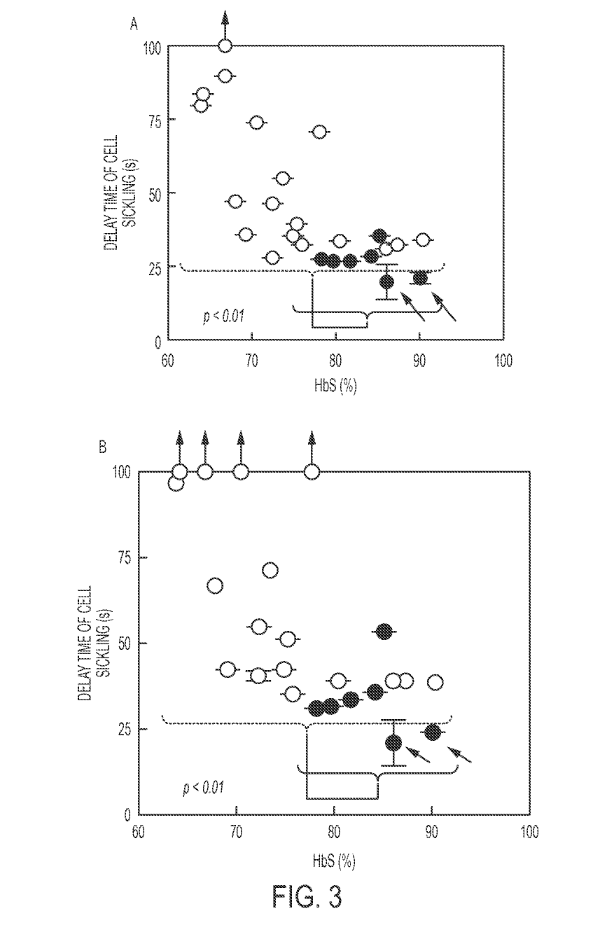

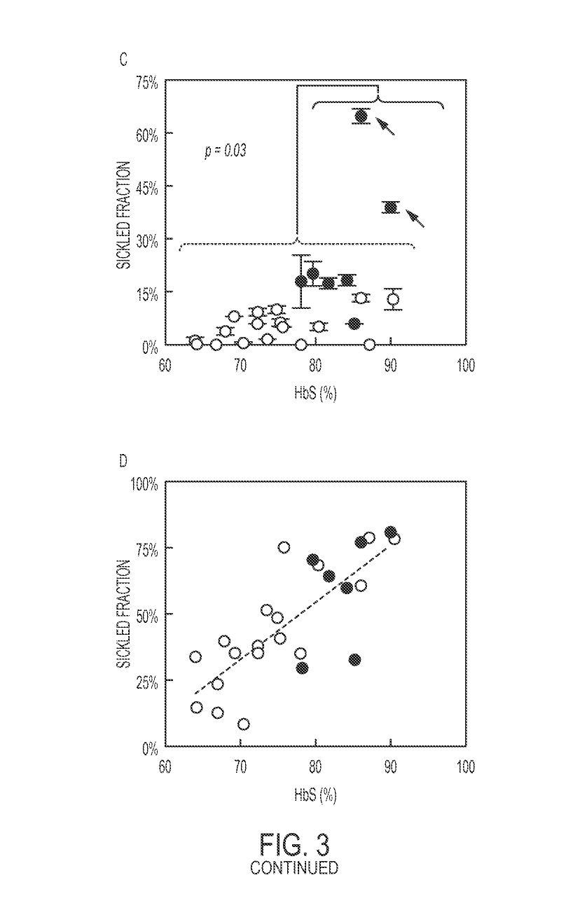

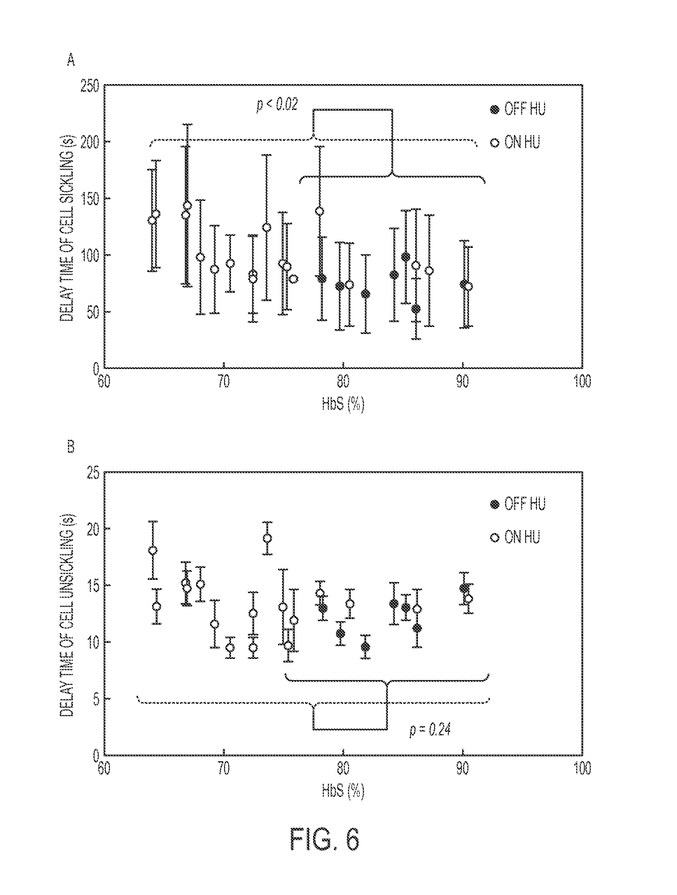

Results of the kinetics of cell sickling were plotted against the % HbS of individual patients (FIG. 3A-B). The delay time of sickling was greater than 25 s for RBCs (i.e. for 5% sickled fraction) for most of the samples in the study (FIG. 3A). The delay time of sickling for the on-HU group was significantly longer than that for the off-HU group (p<0.01). Within the on-HU group, the delay time of sickling (for 5% sickle fraction) varied from 28 s to 100 s, suggesting a difference in the efficacy of HU among different patients. Similar trends were observed at a higher sickled fraction (10%, FIG. 3B). Six cases showed significantly longer delay times (>60 s) than the others, suggesting possible beneficial effect of HU therapy. The two cases with the shortest delay time (less than 25 s) of cell sickling, marked by arrows, suggest relatively higher risk for vaso-occlusion. For the sickled fraction of each sample reaching its saturated level under long-term DeOxy state, delay time of cell sickling varied widely within the same patient and among different patients. The influence of HU therapy was statistically significant for the sickling process (p<0.02) (FIG. 6A). The distribution of delay times of cell unsickling seemed to be random among different patients and no significant difference was found between the on-HU and the off-HU groups (p=0.24, FIG. 6B).

Under the short-term DeOxy state, the maximum sickled fraction for all on-HU samples was below 15%, which was significantly lower than that for the off-HU group (p=0.03, FIG. 3C). Within the off-HU group, the sickled fraction was highly variable among patients, ranging from less than 10% to over 60%. The two outliers with the most severely shortened delay time results showed consistency with the highest sickled fractions (FIG. 3A-C). On the other hand, during the long-term DeOxy state, the maximum sickled fraction showed a strong positive correlation with the HbS level (Pearson's correlation coefficient, R=0.79, p<0.001, FIG. 3D). The levels of sickled fractions under short-term and long-term DeOxy states are comparable with a previous in vitro sickling study (35) under extended DeOxy time (from 1 to 5 h of incubation under 4% O₂). The discrepancy in DeOxy time may be due to the rapid O₂ exchange in cell suspension using our microfluidic system than using the static DeOxy incubation system in the earlier study (35). The result of kinetics of cell sickling correlating with HbF level had a relatively weaker trend in the opposite direction than that with HbS level (e.g. R=0.55, p=0.004 for the sickled fraction under long-term DeOxy state).

### Individual Sickle RBC Rheology.

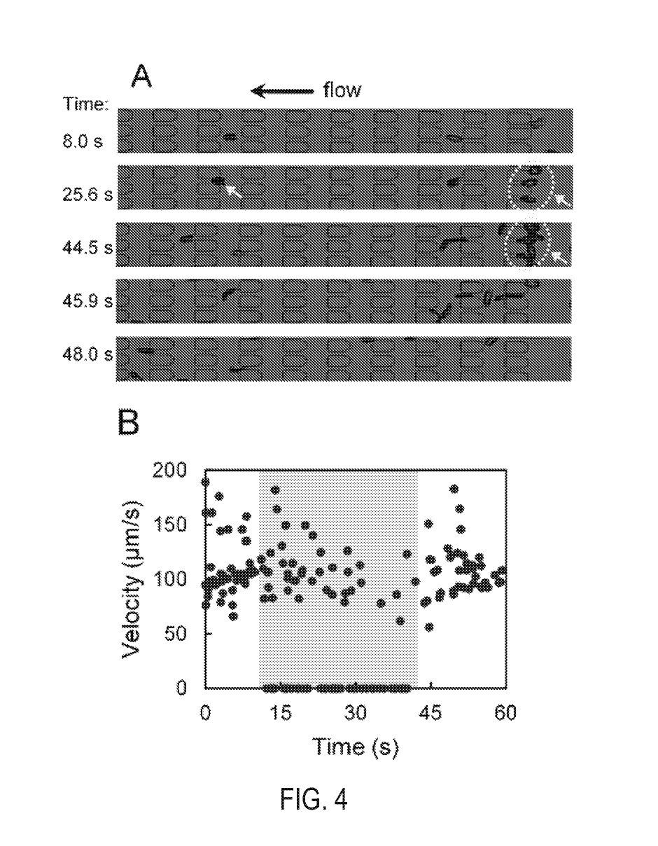

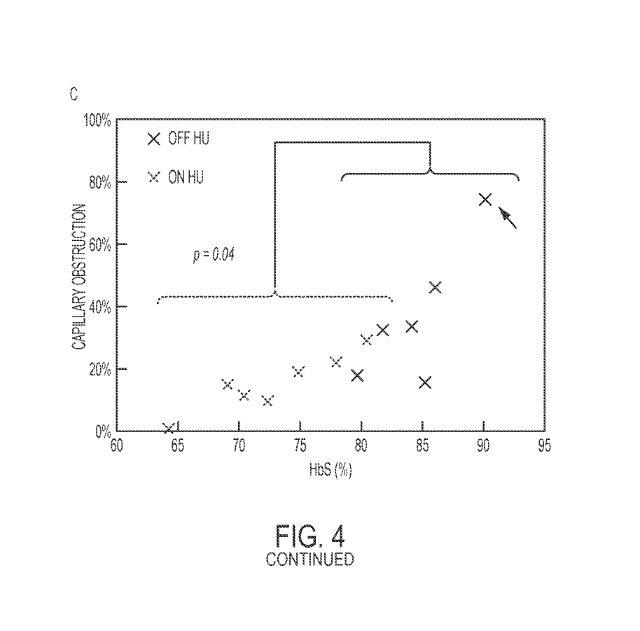

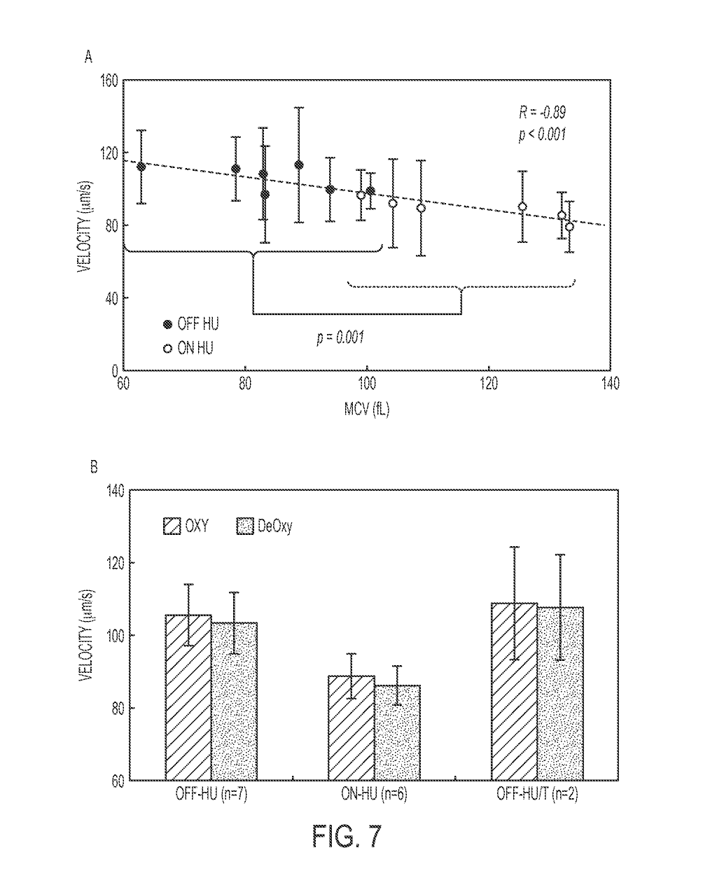

Individual sickle RBC rheology was examined, at a given pressure differential and with a short-term transient hypoxia, as a potential diagnostic indicator of risk for vaso-occlusion. Sickle RBCs were deformable during the initial 12 s (O₂ concentration>5%). Here deformability denotes the ability of the cell to successfully traverse the 4 μm-wide micro-gates. When the O₂ concentration was reduced to less than 5%, the RBCs undergoing sickling were unable to traverse the micro-gates, thereby causing obstruction to RBC flow. With ReOxy, the obstructed RBCs recovered their shape and deformability, and flow was resumed. The velocity of sickle RBCs was then quantified as the average speed over 5 micro-gates for the individual RBCs travelling through the periodic micro-gates. A representative distribution of cell velocities in response to transient hypoxia is shown (FIG. 4B). The velocity of individual sickle RBCs varied widely in the same patient and among different patients. Significant correlation was found, between cell velocity and the mean corpuscular volume (MCV, Pearson's R=−0.89, p<0.001) (see Single cell rheology in Materials and Methods, FIG. 7A-B). The capillary obstruction ratio, was defined as the fraction of total number of cells that were blocked at the micro-gates during the DeOxy state. Sickle cell capillary obstruction ratio, measured on 7 on-HU and 7 off-HU patient samples, increased with HbS concentration (FIG. 4C), similar to that seen with sickled fraction. In general, the on-HU group exhibited significantly lower capillary obstruction ratio than the off-HU group (p=0.04). A severe case was identified with the highest capillary obstruction ratio and marked by an arrow in the figure.

### The Role of Cell Density.

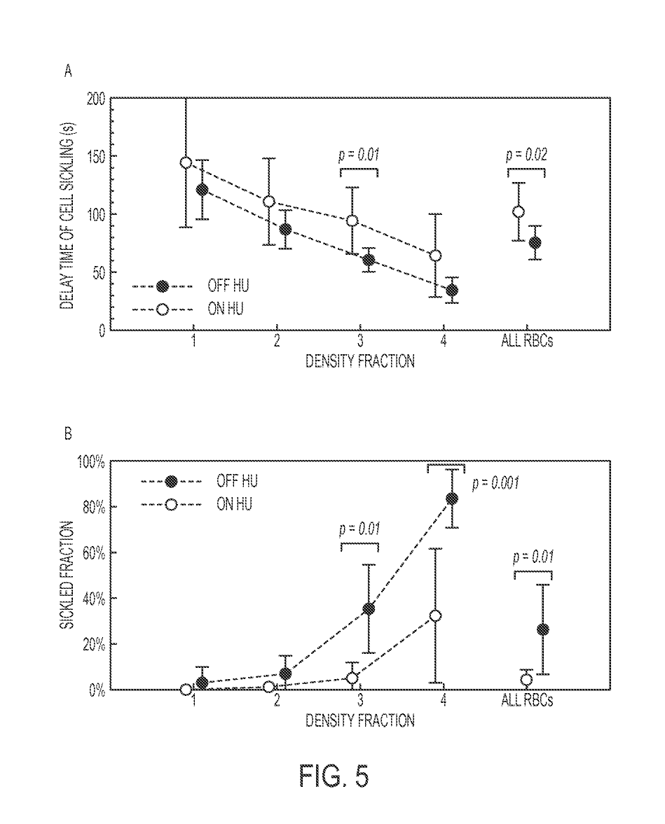

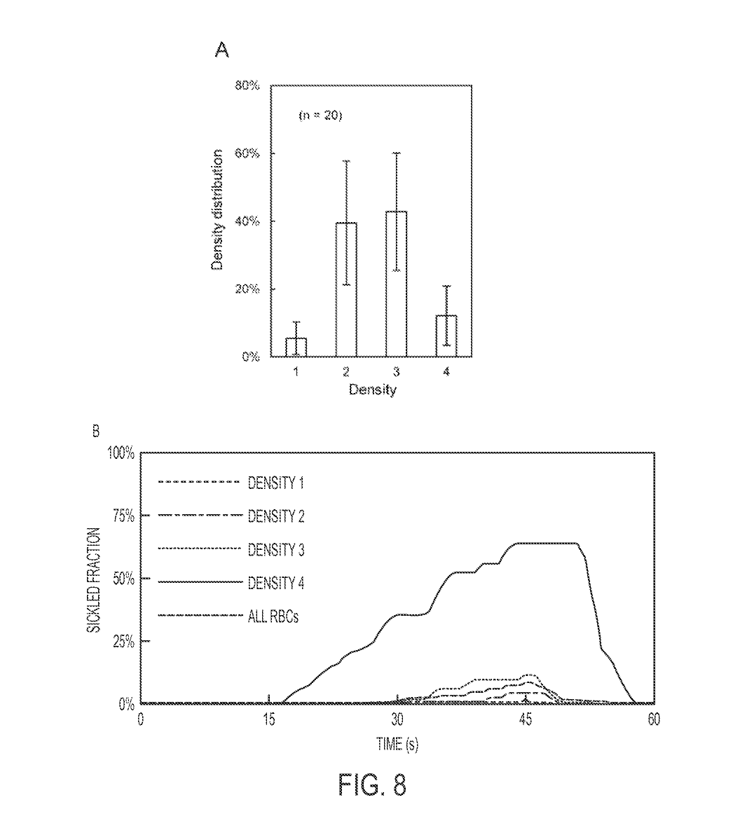

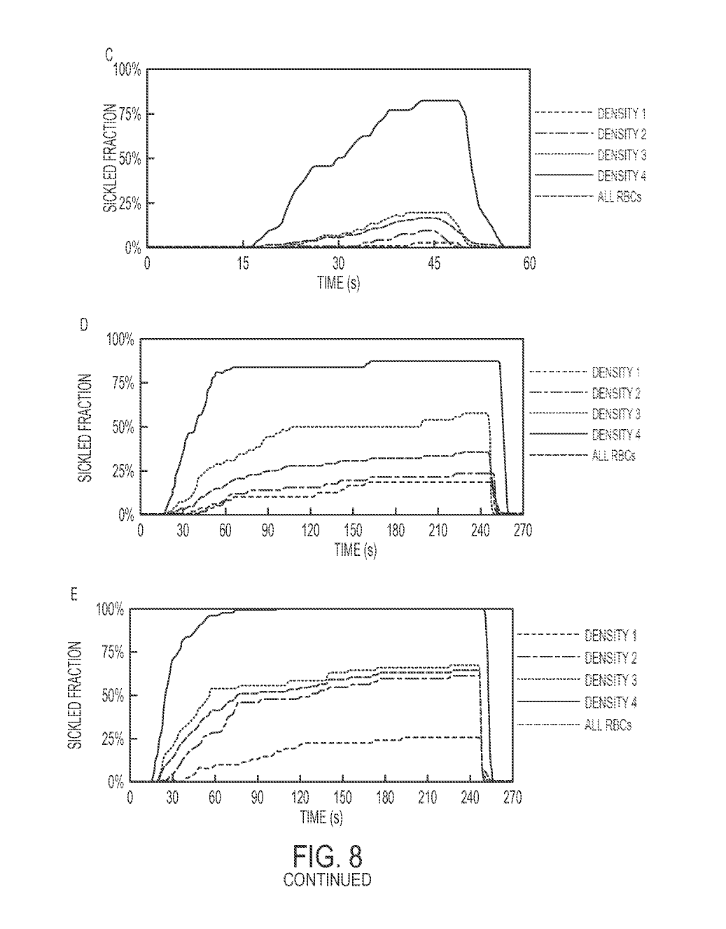

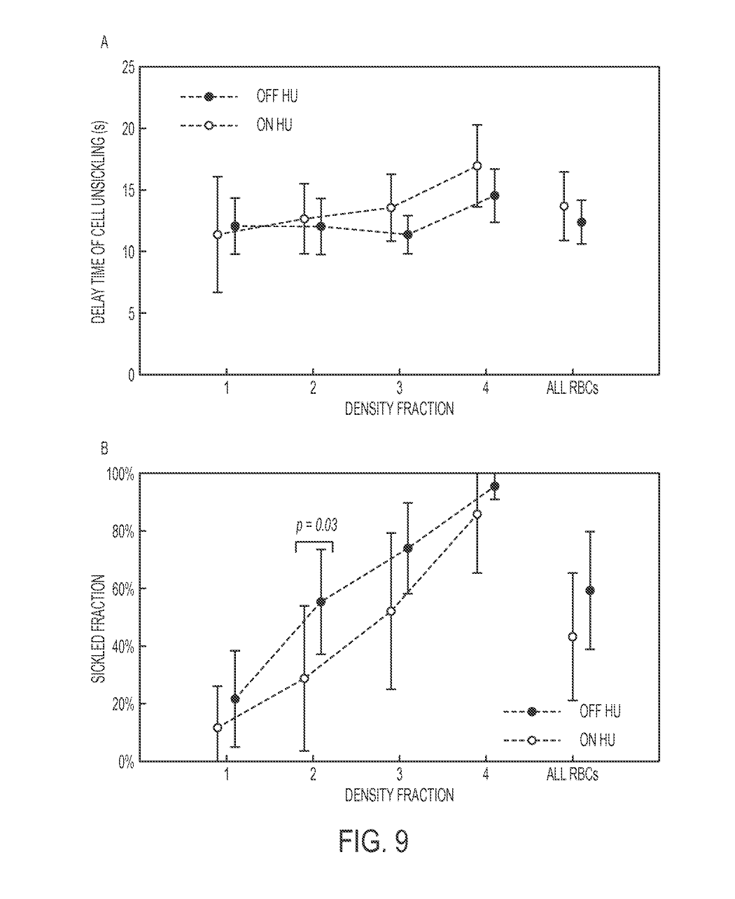

Previous studies demonstrated that sickle RBCs have a broad range of cell density from 1.085 g/ml to 1.146 g/ml (36-38). In order to quantify the effects of cell density on the kinetics of cell sickling and unsickling, we categorized sickle RBCs into four populations with average cell densities of 1.086±0.005 g/ml (Density 1), 1.095±0.005 g/ml (Density 2), 1.105±0.005 g/ml (Density 3), and >1.111 g/ml (Density 4) for 20 SCD samples from 6 off-HU patients and 14 on-HU patients. The majority of sickle RBCs fell within Density 2 and Density 3 (FIG. 8A). We noticed a significant difference in sickling growth curve among different density populations of individual blood samples under both short-term and long-term DeOxy states (FIGS. 8B and C). Results of delay time of cell sickling and sickled fraction were examined along with cell density and the patient's HU status. Delay time of cell sickling decreased with cell density (FIG. 5A), which can be rationalized by the hydration state of the cells (39, 40). The mean delay time of cell sickling for the on-HU cases were statistically higher than off-HU cases (p<0.02). A marked extension in the delay time of cell sickling was seen for Densities 3 and 4 with HU therapy (p=0.01 and p=0.06). The overall delay time for unsickling did not vary significantly among Densities 1 through 3, and between the on-HU and off-HU groups (FIG. 9A).

The maximum sickled fraction showed a strong correlation with cell density disregarding the patient's HU status or hypoxia duration (FIG. 5B and FIG. 9B). This observation is consistent with reported correlation between HbS concentration and polymerization kinetics (41, 42). Under short-term hypoxia, HU therapy significantly suppressed sickled fraction, particularly in Densities 3 and 4 (p=0.01 and p=0.001, respectively).

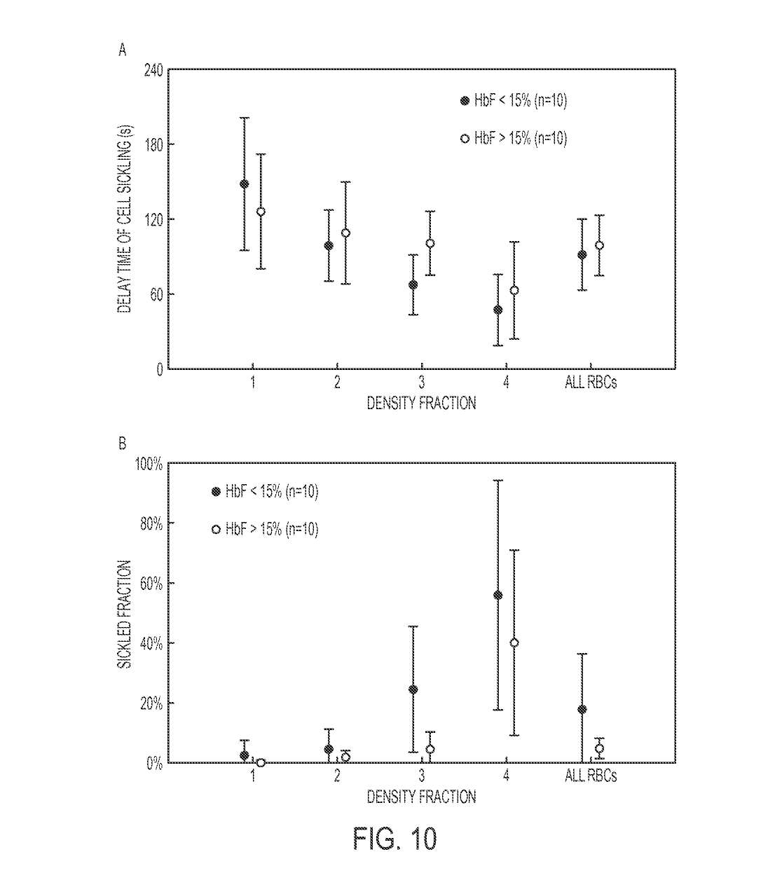

The effects of HbF fractions on density-dependence of the cell sickling kinetics show that the differences between low HbF group (% HbF<15%, n=10) and high HbF group (% HbF>15%, n=10) were not as significant as those between on-HU and off-HU groups (FIG. 10A-B).

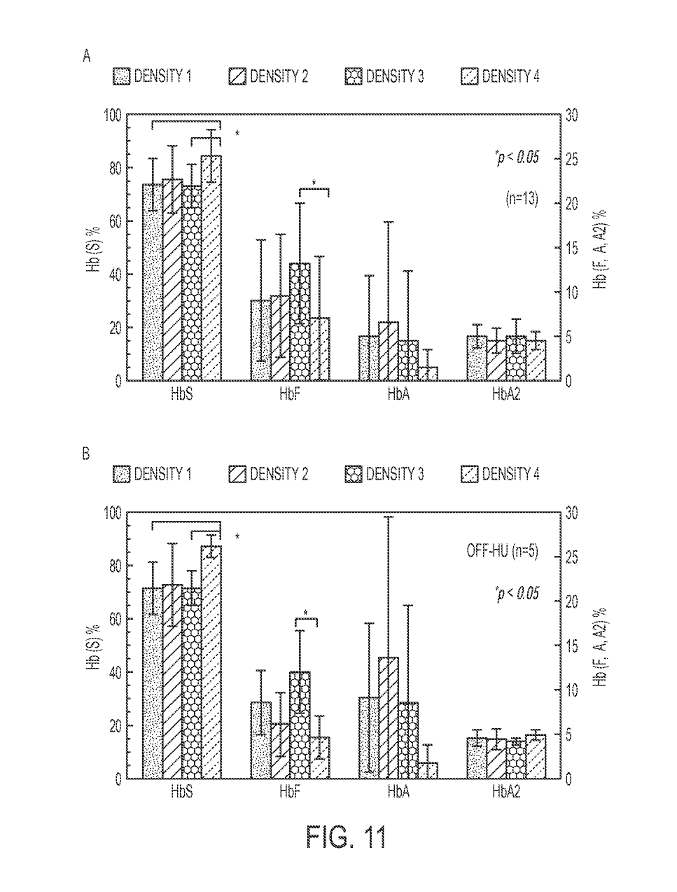

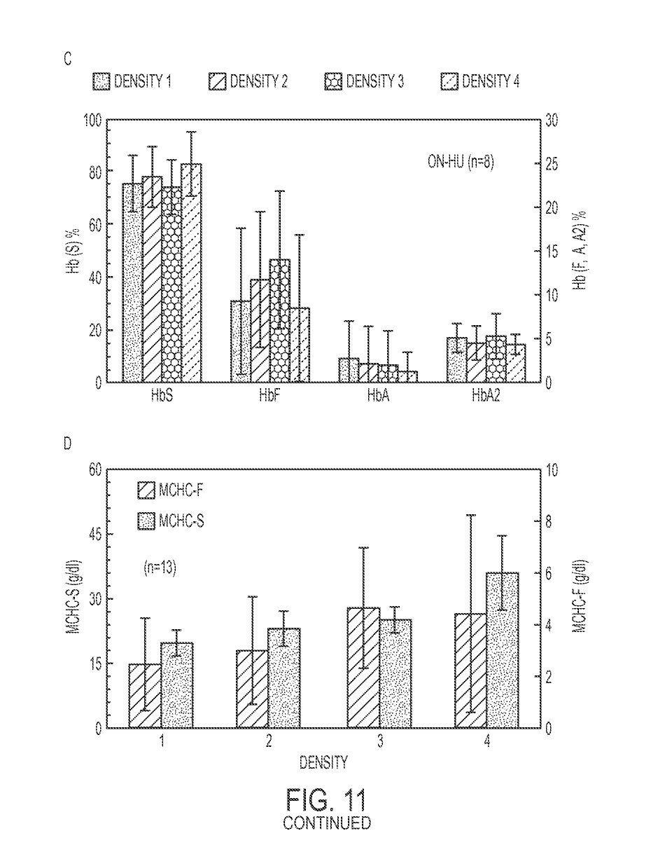

The distribution of Hb types in the density-separated populations was obtained through high performance liquid chromatography (HPLC). The results of 13 patient samples (5 off-HU HU and 8 on-HU) with HbS levels ranging from 66.8% to 90.4% revealed (FIG. 11A-B) that higher levels of HbS and lower levels of HbF in Density 4 than other lighter density populations. This observation is consistent with reports, that dense cells have higher HbS level and lower HbF level than lighter cells (43), and that dense cells have lower HbF levels than all RBCs (44). Surprisingly, there was no significant difference among the three lighter populations for all four Hb types, i.e. HbS, HbF, HbA, and HbA2 (FIG. 11A). The trends for Hb type vs. cell density were quite similar in both off-HU (n=5) and on-HU (n=8) groups (FIGS. 11B and C). This information seems to contradict the strong correlation of cell sickling with cell density, as it has already been demonstrated that cell sickling is highly dependent on % HbS. To better elucidate this result, we established two parameters to take into account both hydration state and Hb content, including mean intracellular HbS concentration, MCHC-S and mean intracellular HbF concentration, MCHC-F (see MCHC in Materials and Methods Text). The distribution of MCHC-S increased with cell density (FIG. 11D). Density 4 had a high MCHC-S value due to the joint effects of high % HbS and the high MCHC value.

### Discussion of Results.

Shape change is a reliable marker for cell sickling in hypoxia-induced sickled RBCs. Through imaging flow cytometry, this shape change is highly correlated with the existence of intracellular HbS polymers identified by transmission electron microcopy (45). Our hypoxia assay is expected to have a higher efficacy for identifying sickled RBCs as it can incorporate another visual characteristic, cell texture, in addition to changes in cell morphology. The majority of sickled cells (density fractions 1 to 3) had apparent shape change. Very few sickled cells, especially in Density 4 showed little or no apparent shape change, but notable changes in cell texture, sharing similar features to the ones at rapid DeOxy rates by reducing agents (25, 31).

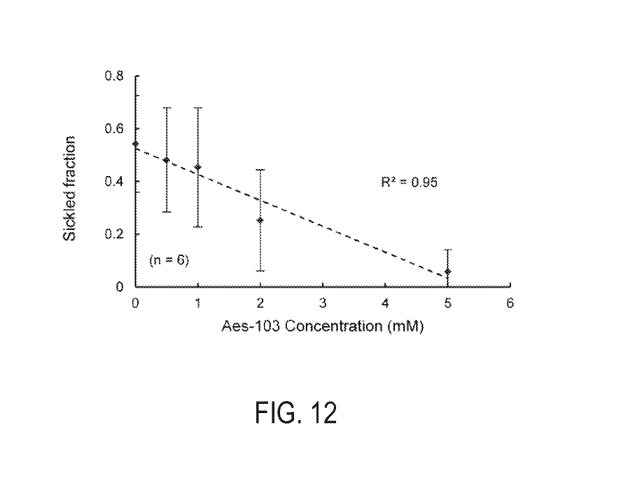

The kinetics of cell sickling was markedly affected by HU therapy, including delay time of cell sickling (p<0.01 for 5% and 10% of sickled fractions; p<0.02 for saturated sickled fraction) and maximum sickled ratio under short-term hypoxia state (p=0.03). This analysis highlighted the beneficial effects of HU therapy on the DeOxy sickle RBCs. These results are consistent with previous clinical reports of disease amelioration through the stimulation of HbF synthesis (46-49). Additionally, we identified outlier patient samples (marked by arrows in FIGS. 3A-C and 4B) that showed the most abnormal results in our assays, including shortest delay time of cell sickling, highest sickled fraction, and highest capillary obstruction ratio, all suggesting high risk for vaso-occlusion. Hematological measurements indicated these two patient samples as severe SCD, according to a genotype-based disease severity classification (50). Our analysis also indicate that HbF levels do not completely account for the kinetics of cell sickling, including the maximum sickled fraction (R=−0.4, p=0.05 for short-term hypoxia state, and R=−0.55, p=0.005 for long-term hypoxia state) and the delay time of cell sickling (R=0.35, p=0. 08 at a low sickled fraction 5%). These observations are consistent with studies indicating only partial correlation between HbF fraction and painful crises (16, 20, 22). The large variations in delay time of cell sickling in on-HU group could correlate with additional outcomes from HU therapy besides HbF induction (51, 52). Therefore, our analysis could offer a unique route to develop a supplementary tool at a cellular level, beyond current hematological assays (53), to evaluate the response to HU and other anti-sickling drugs for individual SCD patients. An example of this is found in Aes-103, (5-hydroxymethylfurfural, 5-HMF) that is currently in phase II clinical trials in SCD patients. The sickled fraction after a long-term hypoxia in sickle RBCs incubated with Aes-103 in vitro showed a strong correlation with the drug concentration (see Anti-sickling drug in Materials and Methods Text, FIG. 12).

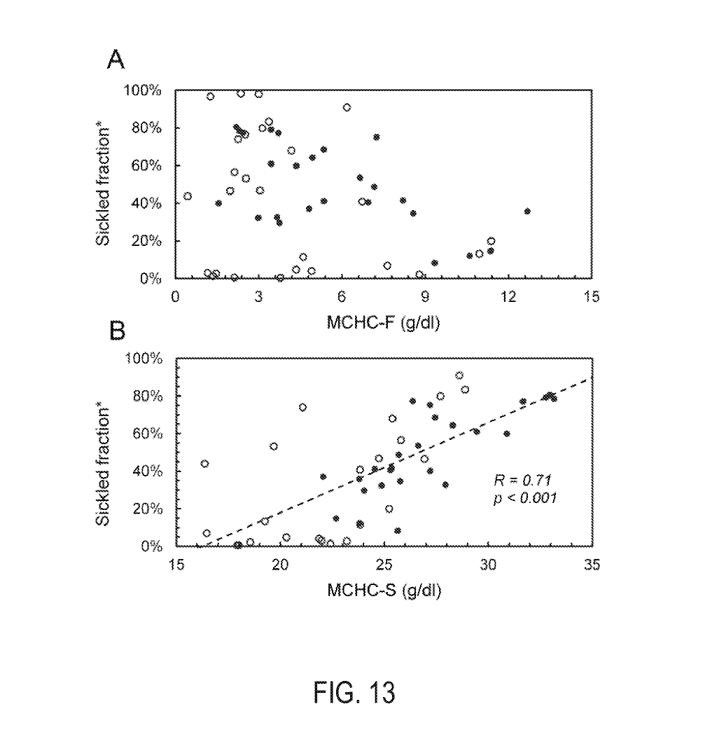

Further analysis of sickling considered hydration state and Hb types. There was no correlation of sickled fraction* (see Effective sickled fraction in Materials and Methods Text, FIG. 13A-B) with MCHC-F (R=0.17, p=0.22) but strong correlation with MCHC-S (R=0.71, p<0.001). These observations indicate that clinical hematological information alone cannot be used to evaluate the cell sickling events in vitro. Further analysis showed a lack of correlation between the sickled fraction* and MCHC-S/F (by multiplying the MCHC value with the ratio of % HbS to % HbF), suggesting MCHC-S is a determinant factor in cell sickling in vitro. These results also imply that when investigating the influence of HbF, the average concentration of HbF in a cell population is less important than the HbF content in individual RBCs (51). This interpretation is supported by an ex vivo study showing incomplete resistance of F-cells in hypoxia-induced sickling (54).

The mean velocity of individual sickle RBCs is an integrative measure modulated by cell size, shape, intracellular viscosity, and membrane deformability, and it could potentially serve as a direct indicator of the ability of cells to transit in capillaries. The opposing effects of elevated cell size (55) and increased membrane deformability (56) due to HU therapy both influence cell traversal through micro-gates. Individual cell velocity was strongly correlated with cell volume (R=−0.89, p<0.001) instead of other hematological measurements (e.g. % HbS, HCT, and MCHC). Cell shape played an important role in transit, especially for the irreversibly sickled cells in the off-HU cases. Additionally, we found that the velocity of deformable cells under the DeOxy state was lower than for cells under the Oxy state, disregarding the influences of HU therapy and transfusion (FIG. 7A-B). This discrepancy may be caused by the increased intracellular viscosity from HbS polymerization and the influence of the degree of oxygenation on HbA (57). The improved rheological properties of sickle RBCs in vivo could therefore stem from the elevated numbers of F cells and the beneficial effects of HbF in cell sickling (55). Similar trends were found in the relationship between sickled fraction and % HbS (FIG. 3C) and between capillary obstruction and % HbS (FIG. 4C), suggesting that morphologic sickling is likely a primary factor in occlusion in capillaries and small vessels.

Density-dependent kinetics of cell sickling provide quantitative measures of selective adhesion and selective trapping of sickle RBCs (58) in shear flow conditions (59, 60) and in vivo conditions (61). Our observations demonstrated that the lightest cells (Density 1) had the longest delay time of sickling and the lowest sickled fraction. This ensured high probability in maintaining deformability for maximum contact area for adhesion during microcirculation, agreeing well with the adhesive dynamics of single sickle RBCs (62). The densest cells (Density 4) exhibited the shortest delay time for cell sickling, the highest sickled fraction, and the longest delay time for cell unsickling, which may contribute to quick stiffening and ready trapping.

We found that the beneficial effects of HU therapy on sickling kinetics were more evident for the relatively dense populations, in terms of the delay time of cell sickling (p=0.01 for Density 3 and p=0.06 for Density 4, respectively) and the maximum sickled fraction (p=0.01 and p=0.001 for Density 3 and Density 4, respectively). These factors could serve as candidate biomarkers to evaluate the efficacy of HU therapy and to guide the development of new therapeutics.

### Materials and Methods

### Sickle RBC Samples.

Blood samples from 40 SCD patients, including 26 patients with HU therapy (on-HU), 12 patients without HU therapy (off-HU), and 2 patients off-HU but with transfusion (off-HU/T) were collected in EDTA anticoagulant at the National Institutes of Health and Massachusetts

General Hospital, and shipped to MIT on ice and stored at 4° C. All the microfluidics tests were conducted within 3 days of blood drawn. For cell sickling/unsickling tests, we utilized 25 samples (18 on-HU and 7 off-HU). For the single cell rheology test, we utilized 16 samples, including 7 on-HU, 7 off-HU and 2 off-HU/T. For the study of cell density, we utilized 20 samples, including 14 on-HU and 6 off-HU. Another 13 samples (8 on-HU and 5 off-HU) were utilized for the HPLC characterization. For the Aes-103 testing, blood samples from 3 on-HU and 3 off-HU patients were incubated with Aes-103 at different concentrations (0.5, 1, 2, and 5 mM) for one hour at 37° C. before the in vitro sickling test. Sickle RBC fractionation according to cell density was performed by means of a stepwise gradient prepared with Optiprep solution with density adjusted with Dulbecco's Phosphate Buffered Saline (HyClone DPBS, Thermo Scientific) based on the specific gravity. The fractionation gradient was built up with four layers of 2.5 ml Optiprep-DPBS medium of densities of 1.081, 1.091, 1.100 and 1.111 g/ml, respectively. 1 ml blood sample was washed twice with Phosphate Buffered Saline (PBS) at 2000 rpm for 5 minutes at 21° C. and diluted into 70-80% hematocrit. Then the RBC pellet was fully suspended by gentle vortexing and layered on top of the gradient. Cell fractionation was achieved by 30 minutes centrifugation at 2000 rpm and 21° C. The four fractionated populations trapped between the interfaces of successive layers of gradient medium and in the bottom of the tube were carefully collected with 1 ml pipette tip and washed with 5 ml PBS buffer twice to remove gradient residue. The four fractions, Density 1 through Density 4 have mean densities of 1.086±0.005 g/ml, 1.095±0.005 g/ml, 1.105±0.005 g/ml, and >1.111 g/ml, respectively. Fractionated sickle RBCs were then suspended with RPMI-1640 containing 1% w/v Bovine Serum Albumin (BSA) (Sigma-Aldrich, St Louis, Mo.) and stored at 4° C. until shortly before use to avoid metabolic depletion. BSA was used to maintain the cellular livability and prevent cell adhesion to the interior surfaces of microfluidic devices.

### Microfluidic Platform.

Microfluidic devices were designed and fabricated using polydimethylsiloxane (PDMS) casting protocols and bonded to microscope slides. The masters of PDMS channels were fabricated with silicon wafers using standard photo-lithography techniques and followed with two-hour surface passivation using fluorinated silane vapor ((tridecafluoro-1,1,2,2-tetrahydrooctyl)-1-trichlorosilane, T2492-KG, United Chemical Technologies). O₂ concentration in the cell channel was controlled by the gas flow in the gas channel. The transient hypoxia condition was created by switching between two gas mixtures, including a gas mixture of 5% CO₂, 20% O₂ with N₂ balance for an initial oxygenation and reoxygenation and a gas mixture of 5% CO₂, 2% O₂ with N₂ balance for deoxygenation. Two reservoirs (1.5 mm diameter and 2 mm deep) for cell buffer exchange were fabricated 1.2 mm away from the obstacles and connected to the external hydraulic columns via flexible TYGON microbore tubing (0.020″ ID×0.060″0D, not shown in the figure). Prior to the rheology test, the microfluidic devices were degassed for at least 15 minutes before filling with working medium to improve wetting and prevent air bubble trapping.

### Experimental Conditions.

Experiments were performed on a Zeiss Axiovert 200 inverted microscope (Carl Zeiss Inc., Thornwood, N.Y.) using a halogen source (100 W). Microfluidic devices were enclosed in a heating incubator (Ibidi heating system) with temperature maintained at 37° C. for both cell sickling and rheology measurements. The temperature state of the cell buffer within the microfluidic channel was calibrated with a thermocouple considering the mass exchange (gas and cell buffer) prior to experiment. Image of RBCs was enhanced with a 414/46 nm band-pass filter (Semrock). Local O₂ concentration in cell channel was characterized offline using Tris(4,7-diphenyl-1,10-phenanthroline)ruthenium(II) dichloride complex (Ru(dpp)₃C1₂, Sigma-Aldrich), the fluorescence of which is strongly reduced by molecular O₂ due to dynamic quenching. As Ru(dpp)₃C1₂ is water insoluble, it was dissolved in Acetone-RPMI (volume ratio of 1:2) at 0.8 mg/ml and injected into the cell channel. Luminescence was measured at an emission wavelength of 488 nm. Short-term and long-term hypoxia conditions held the durations of DeOxy state (O₂ concentration<5%) about 25 s and 220 s, respectively. The division of 5% was selected because sickle RBCs start to sickle when O₂ was lower than this point.

### HPLC.

The relative proportions of HbS, HbF, HbA, and HbA₂ of density-separated cell populations were obtained via HPLC performed at Brigham and Women's Hospital (Boston, Mass.).

### Statistic Study.

All data are expressed as mean±SD. Statistical analyses were performed with OriginPro 8. A two-sample t-test between measurements of samples from on-HU patients and off-HU patients was used to generate the p-values with equal variance not assumed. Correlation analyses between the biophysical measurements and the hematological values were performed using Pearson's correlation.

### Patient's HU Status.

It was ascertained that on-HU patients were prescribed HU therapy, and treated for several months to years. Dosage is normally started low and gradually increased to maximum tolerated dose without side effects. It was also known that off-HU patients were not prescribed HU.

### MCHC Estimation.

Based the linear correlation between cell density and MCHC value (1), we calculated the values of MCHC for each density fractions to be 27.3, 30.9, 34.9, and >37.4 g/dL for Density 1, 2, 3 and 4, respectively. Further HPLC analysis in combination with cell sickling confirmed a lack of correlation between HbF concentration and the sickled fraction of the DeOxy cells. Interestingly, HPLC analysis revealed that levels of HbS and other hemoglobin types, HbF, HbA, and HbA2 did not vary significantly among the density-separated populations except the dense cell populations (FIG. 6A-B), while the influence of cell density and HbS concentration were well pronounced in the microfluidic tests (FIG. 4B). These findings highlighted the importance of hydration state in cell sickling. We then utilized MCHC-S and MCHC-F to take into accounts both hydration state and Hb content. The values were obtained by multiplying the MCHC value with % HbS or % HbF.

### Single Cell Rheology.

Significant correlation was found, between cell velocity and the mean corpuscular volume (MCV, Pearson's R=−0.89, p<0.001). No notable correlation was found between cell velocity and other hematological parameters (e.g., MCHC, HCT, % HbS, % HbF, % HbA, % HbA2, WBC). MCV value is in the range of 63 to 101 fl for the off-HU group and from 99 to 133 fl for the on-HU group in the study. Cell velocities of the on-HU group were thus lower than those for the off-HU group, which is ascribed to elevated cell volume resulting from HU therapy. Cell velocities of two cases of off-HU with transfusion (off-HU/T, with HbS of 43.5% and 44.3%, respectively) were essentially same as those for the off-HU cases without transfusion, mainly because of similar MCV levels (FIG. 2B).

### Anti-Sickling Drug.

Aes-103 (5-hydroxymethylfurfural, 5-HMF) can stabilize the R-state and increase the oxygen affinity of hemoglobin. Its anti-sickling effects have been demonstrated in SCD under both in vitro and in vivo conditions (2-4). Here we evaluated our microfluidic assay by quantifying the anti-sickling effect of Aes-103 at millimolar concentrations (mM) on 3 on-HU and 3 off-HU patient samples (% HbS ranges from 69.2% to 90.1%). The distribution of sickled fractions does not completely correlate with the patient's HU status. In the absence of Aes-103, the sickled fractions varied from 34% to 73% (Mean±SD: 54%±18%). With the presence of Aes-103, the sickled fraction decreased with drug concentration with R²=0.95 for a linear regression (FIG. 7A-B). This trend is consistent with a previous study (2).

### Effective Sickled Fraction (Sickled Fraction*).

To exclude the influence of HbA, an effective sickled fraction (sickled fraction*) was used based on the sickled fraction divided by 1-HbA concentration.

### Example 2

### Quantification of Anti-Sickling Effect of Aes-103 in Sickle Cell Disease Using an In Vitro Microfluidic Assay

### Introduction

Under hypoxic conditions, sickle hemoglobin (HbS) polymerizes, causing morphologic distortion (sickling) of red blood cells (RBCs) in sickle cell disease (SCD). Aes-103 (5-hydroxymethylfurfural, 5-HMF) can stabilize the R-state and increase the oxygen affinity of hemoglobin, inhibiting the intracellular polymerization of HbS. Using a microfluidics-based hypoxia assay, we were able to track sickling of individual cells and quantify the anti-sickling effect of Aes-103 at millimolar (mM) levels in blood from SCD patients on hydroxyurea treatment (on-HU) and not on hydroxyurea treatment (off-HU).

### Methods

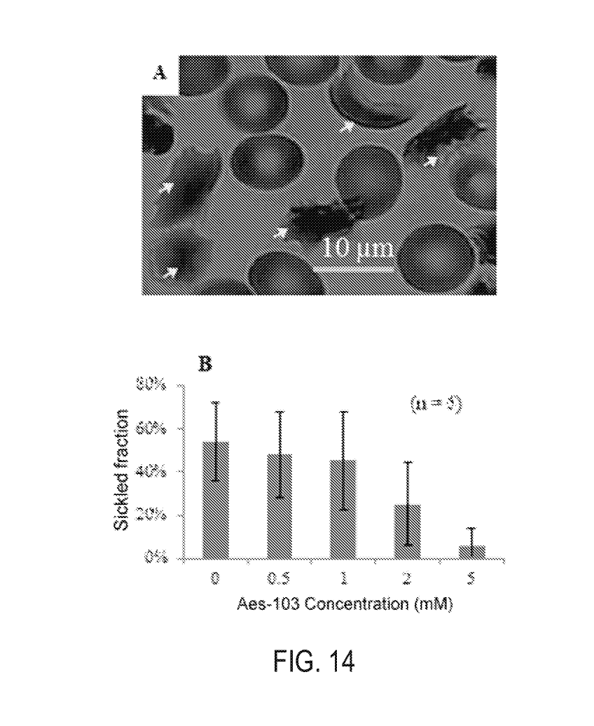

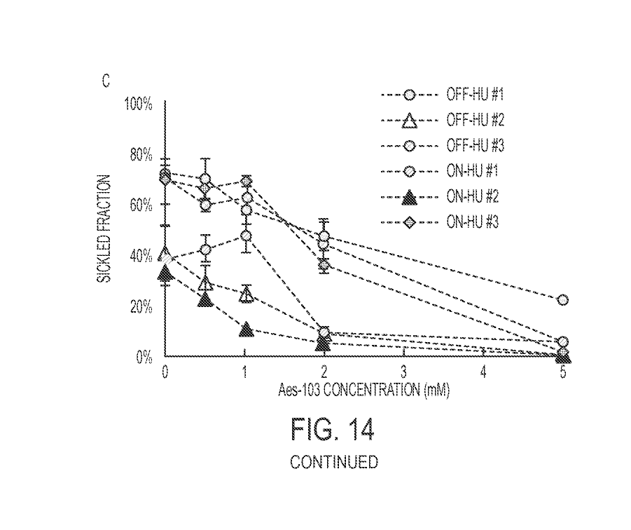

A microfluidic assay was developed that utilizes a gas permeable polydimethylsiloxane (PDMS) film 150 μm in thickness, to create a severe hypoxia microenvironment in a 5 μm deep chamber to measure cell sickling in vitro at 37° C. The hypoxia condition was 5 minutes in total, consisting of an initial oxygen-rich stage (20% O₂), a transient deoxygenating stage (O₂ concentration decreased to 5% within 15 second), and a steady-stage stage (O₂ concentration decreased further and maintained at 2% for the rest of time). Blood samples from 3 on-HU and 3 off-HU patients were incubated with Aes-103 at concentrations of 0.5, 1, 2, and 5 mM for one hour at 37° C., washed with Phosphate Buffered Saline and suspended in RPMI-1640 containing 1% w/v Bovine Serum Albumin for in vitro testing. Sickle RBCs undergoing sickling typically form spiky edges and a dark coarse texture due to intracellular HbS polymerization visually enhanced by a bandpass filter (FIG. 14A). The anti-sickling effect of Aes-103 was then quantified by the maximum sickled fraction (fraction of all RBCs that were morphologically distorted) under the hypoxia condition.

### Results.

In the absence of Aes-103, the sickled fractions varied from 34% to 73% (Mean±SD: 54%±18%). With the presence of Aes-103, the mean sickled fraction decreased with drug concentration (FIG. 14B), which can be well fitted with linear regression (R²=0.95). With 2 mM Aes-103 incubation, each patient sample showed a significant decrease in cell sickling from its baseline. Addition of Aes-103 at 5 mM concentration prevented majority of RBCs from sickling (sickled fraction <5%). The sickled fraction of one patient sample was nearly zero. The distribution of sickled fractions does not completely correlate with the patient's HU status in this limited sample size (FIG. 14C). We also observed that hypoxia-induced sickling at baseline showed an apparent bimodal distribution, although the slope of response to Aes-103 concentration was similar.

### Conclusions.

Our microfluidic assay enabled a rapid, quantitative characterization of cell sickling in vitro within a few minutes and using a single drop of whole blood patient sample. We confirmed the anti-sickling efficacy of Aes-103 for both on-HU and off-HU patient samples in a dosage-dependent manner. This assay is useful for drug development and monitoring for in vivo effect of potential anti-sickling therapeutics.

### Example 3

### “Memory” in Cell Sickling During Continuing Deoxygenation and Oxygenation Cycles

### Summary

The disclosure provides an in vitro study of repetitive sickling and unsickling of freely suspended red blood cells (RBCs) from patients with sickle cell disease using a microfluidic hypoxia assay. This assay enables a real time observation and measurement of morphologic distortion and recovery of individual sickle RBCs under continuing deoxygenation and oxygenation cycles. Cell deformity may initiate randomly at cell edges and away from the dimple region and then branch through the entire cell, indicating that formation of primary HbS fibers may be enhanced at those sites on the cell membrane. Morphology of cell deformity demonstrates that repetitive deoxygenation did not induce identical deformity in the same individual RBCs. Kinetics of cell sickling implies a “memory” in cell sickling event. Evidence supporting such “memory” include, for example, the increased sickled fraction and reduced delay time of cell sickling along with deoxygenation and oxygenation cycles. Methods provided herein can be used as an accelerated damage model of sickle cells in response to repetitive hypoxia cycles with implications of in vivo pathophysiological processes of cell sickling and unsickling in circulation.

### Introduction

Intracellular polymerization of deoxygenated sickle hemoglobin (HbS) may cause poorly deformable, distorted red blood cells (RBCs) in sickle cell disease (SCD). This process is known as cell sickling and plays a pathophysiologic role in SCD. HbS polymerization may be associated with cell dehydration and increased cell density (higher HbS concentration), which can further accelerate HbS polymerization and cell sickling.

Repetitive deoxygenation (DeOxy)-Oxygenation (Oxy) cycles exerted on sickle RBCs in blood circulation may induce cyclic polymerization-depolymerization of HbS and consequent cell sickling-unsickling. These repeated processes may cause cell dehydration, poor deformability, hemolysis, and can be associated with deleterious effects on the vasculature and impaired blood flow. Additional shear stresses exerted on sickle RBCs from blood flow in varied vasculatures may pose severe cyclic mechanical loading to cell membrane and may accelerate the damage process of sickle RBCs. Studies where sickle RBCs were challenged by 100% O₂ and 100% N₂ have shown that RBCs did not exhibit identical sickle deformities. In one study, cell sickling was triggered by photodissociation of CO bonded-hemoglobin and exhibited a ‘memory’ in cell transformation of its previous cycles or cycles. A platform, including any of the devices provided herein, capable of controlling hypoxia to mimic continuing DeOxy-Oxy cycles in blood flow may be useful for providing a basis for the study of intracellular HbS polymerization-depolymerization and associated cell sickling-unsickling and varying blood rheology in SCD.

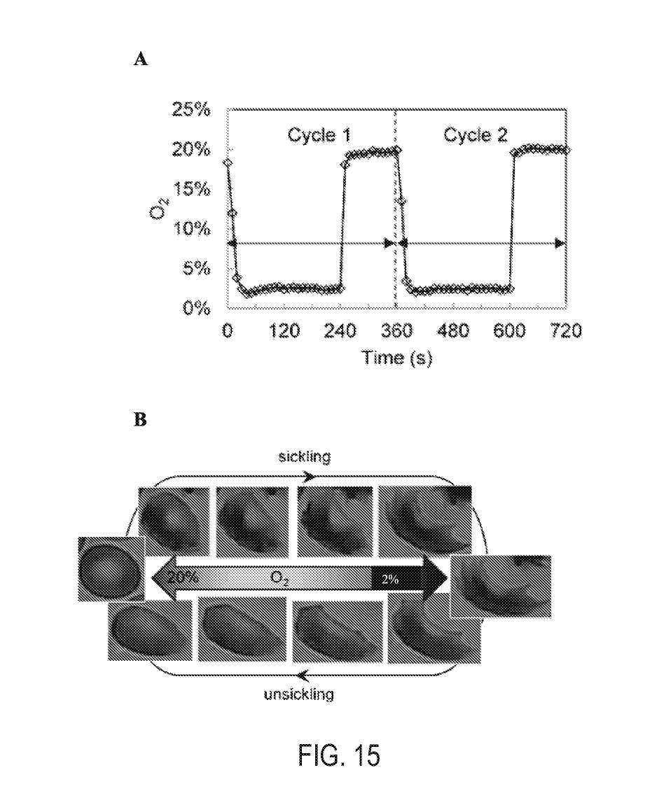

Microfluidics provides a platform in controlled hypoxic microenvironment for the study of cell sickling at single cell level. Described herein is in vitro study of repetitive sickling and unsickling of sickle RBCs that are freely suspended in a microfluidic hypoxia assay. Morphologic transformation of RBCs exposed to transient hypoxia in a microfluidic assay demonstrated a connection between growth of intracellular HbS polymers by DeOxy and melting by re-oxygenation (ReOxy) (FIG. 15A-B). A time lapse of cell transformation due to sickling-unsickling in response to transient hypoxia indicates in vitro hypoxia-induced cell sickling can start with initiation of intracellular HbS polymerization, for example, at cell edges of the projected images, followed by growth of HbS polymers, and eventually protrusions that severely distort the cell membrane. Associated with intracellular polymerization of HbS is cell deformity, e.g., from a fully relaxed biconcave, disc shape to a fully sickle shape. An in vitro ReOxy-induced cell unsickling can exhibit an opposite process to the cell sickling process. HbS fibers that distort the cell membrane can melt, followed by the dissolution of the initially polymerized HbS.

### Materials and Methods

### RBC Preparation

Blood samples from 6 patients with homozygous SCD and with hydroxyurea therapy were collected in EDTA anticoagulant and stored at 4° C. before measurement. The sample pool has an HbS level varying from 66.8% to 90.4% and a Fetal hemoglobin (HbF) level varying from 6.3% to 29.8%. A volume of 1 ml of each blood sample was washed twice with Phosphate Buffered Saline (PBS) at 2000 rpm for 5 minutes at 21° C. A volume of 5 μl RBCs was carefully pipetted from the pellet and fully suspended by gentle vortexing in 1 ml RPMI-1640 containing 1% w/v Bovine Serum Albumin (BSA).

### Cyclic Hypoxia Assay

A microfluidic hypoxia assay was performed using a double-layer structure, fabricated using standard polydimethylsiloxane (PDMS) casting protocols, bonded to a microscope cover slip. Temperature within the microfluidic device was maintained at 37° C. using a heating incubator (e.g., an Ibidi heating system). Sickle RBC suspension was loaded in the cell channel which was separated by a thin PDMS membrane from the gas channel. O₂ concentration of the freely suspended RBCs was then controlled by exchanging gas flow in the gas channel: fully oxygenated state (Oxy) was created using an oxygen-rich gas mixture (5% CO₂, 20% O₂, and 75% N₂) and deoxygenated state (DeOxy) was created by an oxygen-poor gas mixture (5% CO₂, 2% O₂, and 93% N₂). Repetitive hypoxia was created by switching between these two gas supplies at fixed time intervals, resulting in a 220-second DeOxy state (O₂<5%) and a 140-second Oxy state (O₂ concentration>5%). This design allowed real time tracking of individual RBCs during repetitive DeOxy-Oxy cycles on a Zeiss Axiovert 200 inverted microscope (Carl Zeiss Inc., Thornwood, N.Y.).

### Sickling Analysis

Microscopic video of sickle cells challenged by continuing Oxy-DeOxy states was recorded at 1 frame per second. Cell sickling was identified by morphologic changes of sickle RBCs. The image of RBCs was visually enhanced with a 414/46 nm bandpass filter, which includes the optical absorption spectra for Oxy-hemoglobin and DeOxy-hemoglobin. Sickling analysis was carried out in two aspects, cell deformity and kinetics of cell sickling. Kinetics of cell sickling was quantified by sickled fraction (fraction of cells undergoing morphologic sickling) and delay time of cell sickling (the time elapsed between the initiation of DeOxy and the point when a cell shows optically visible features of morphologic sickling). Time for completion of cell sickling is defined as the time elapsed from the point when a cell exhibits apparent features of cell sickling to the point when that cell exhibits a fully sickled shape (no further morphological change can be observed). Data were expressed as mean±SD. Power law was used for curve fitting.

### Results

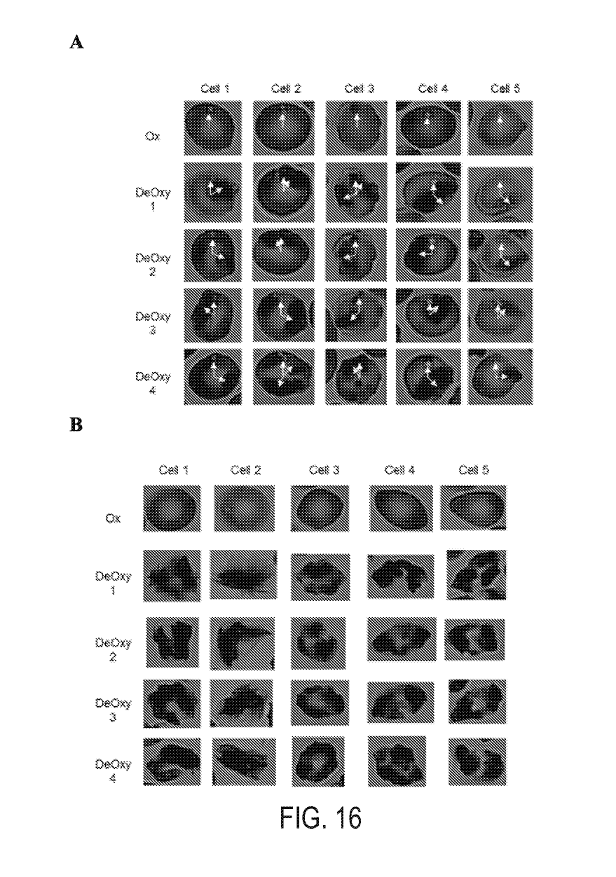

An in vitro hypoxia assay enabled tracking of individual sickle cells during continuing DeOxy and ReOxy cycles. Morphological sickling indicated a random pattern in hypoxia-induced cell deformity, including initial and eventual transformations (FIG. 16A-B). Cell deformity initiated randomly at cell edges based on the individually tracked cells in suspension (FIG. 16A). Cells with minor structural markers (such as spicules or defects on cell membrane) were selected to demonstrate this finding. The arrow pointing strait up indicates the reference orientation of the projected images of selected single cells. The other arrow (or arrows) indicates the initial sites of cell transformation induced by intracellular HbS polymerization. During each cycle, the intracellular HbS polymer strands or clusters (dark regions as shown in the figures) did not share the same orientation. Cell transformation may initiate from single site or multiple sites. Deformity pattern of single sickled cells was random during continuing DeOxy and ReOxy cycles. The data showed repeated sickling and unsickling of freely suspended RBCs under cyclic hypoxia. Representative cells with fully sickled shapes during four consecutive hypoxia cycles are shown in FIG. 16A. The five sickled RBCs showed distinctly different deformity. The same cell did not follow the same pattern in deformation. Additionally, individual severely deformed RBCs fully recovered to their initial relaxed shape after each ReOxy without apparent membrane loss (vesicles) or permanent damages. This demonstrates a lack of “plastic deformation” in cell membrane for freely suspended cells challenged by limited DeOxy cycles.

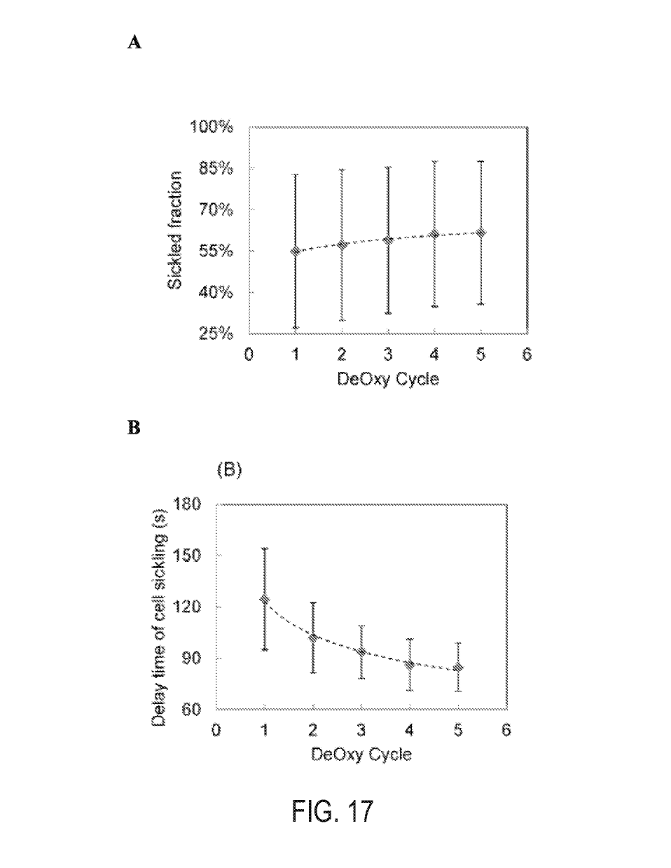

Kinetics of cell sickling was quantified with two parameters, sickled fraction and delay time of cell sickling. To examine the effects of repeated DeOxy and ReOxy on individual sickle RBCs, values of these two parameters for a representative sample were plotted as functions of the DeOxy cycle (FIG. 17A-B). Sickled fraction increased with the hypoxia cycling. This may have been due to the fact that in each Deoxy cycle, newly sickled RBCs were formed in addition to the ones that already sickled in a previous cycle. The delay time of cell sickling decreased with the deoxygenation cycle, indicating an increased sickling rate. Both plots can be fitted with power law functions.

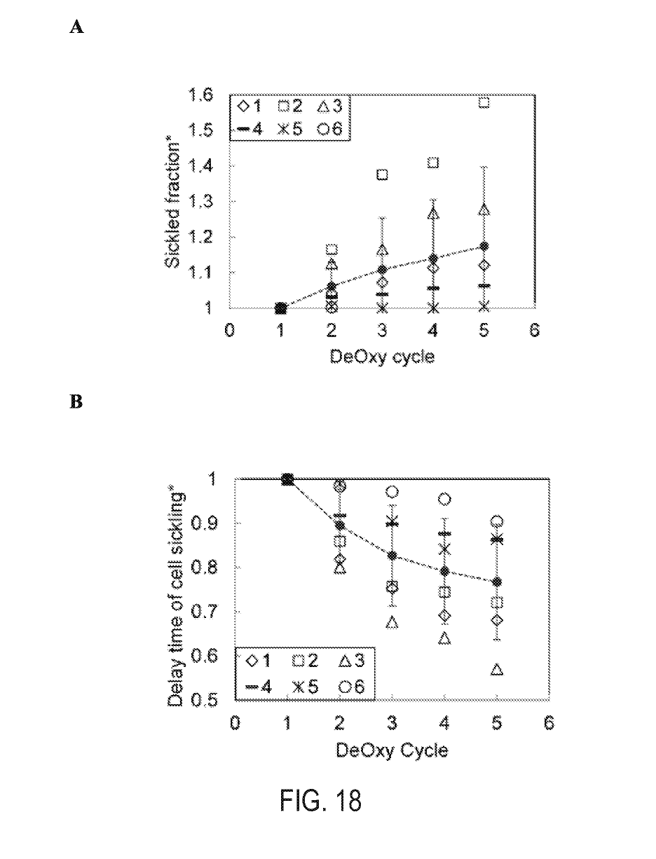

To assess whether the “memory” in cell sickling is present in general, the kinetics of cell sickling of 6 different patient samples were analyzed. Each parameter was normalized to the value measured during the first DeOxy cycle for the specific patient sample and plotted as a function of DeOxy cycle (FIG. 18A-B). Each open symbol represents an individual patient sample and the solid symbols represent the averaged value of all 6 samples. Dashed curves fitted by power law functions indicated a general trend in cell sickling against the DeOxy cycle. Large variations were observed among different patients, indicating heterogeneity in SCD.

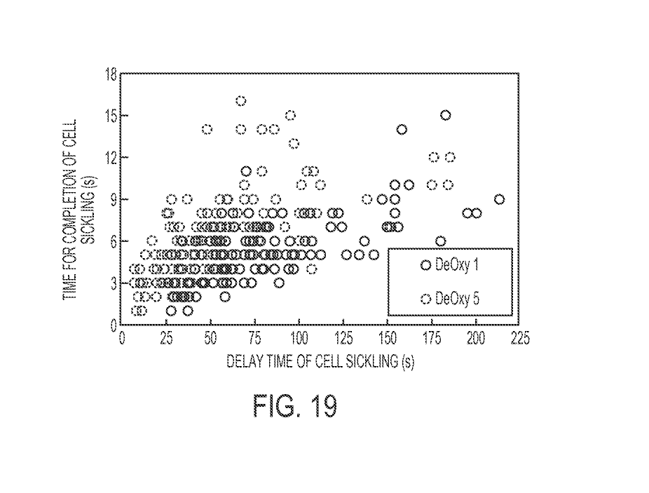

Tracking of 134 different cells randomly selected from different patient samples indicated completion of cell sickling takes place in as short as 1 second to more than 15 seconds (FIG. 19). The average time for completion of cell sickling was of similar level during the five continuing DeOxy and ReOxy cycles and the difference was not statistically different. However, a shift could be observed in the completion time versus delay time of cell sickling for the individual cells. This may be due to the shortened delay time of cell sickling along with the DeOxy cycle.

### Discussion

The data of kinetics of cell sickling provided herein indicate the presence of “memory” in hypoxia-induced cell sickling in vitro. Its presence has been demonstrated in two aspects. First, the sickled fraction increased with the hypoxia cycle. This may be because in each hypoxia cycle, newly sickled cells were formed. The RBCs that had a history of sickling in a previous cycle retained their “memory” and would sickle again. Second, the delay time of cell sickling decreased with the hypoxia cycle. Cell sickling requires intracellular fiber formation through extensive polymer alignment. The time for completion of cell sickling, namely the time elapsed from the first apparent sign of cell deformity to the fully sickled deformity was found to be in a range of less than 1 second to more than 15 second. The overall delay time of cell sickling ranged from less than 30 s to several minutes. These observations highlight the critical role of delay time in preventing most RBCs from sickling during in vivo circulation.

Tracking individual RBC sickling showed that RBC deformation often started at the edges of cell membrane and differed from cycle to cycle. It should be noted that initiation sites were not always associated with visual defects of cell membrane. This observation indicated that formation of primary HbS fibers may be enhanced at those particular sites on the cell membrane, which may also point to possible leak sites with higher K efflux and Na influx. Deformity of individual sickle cells was random during repetitive hypoxia, which may have been due to the randomly branched HbS polymers after the primary polymerization at the particular sites at cell edges. This can be explained by a 2-step mechanism of HbS polymerization and is consistent with the observations of “unpredictable polymerization of HbS” under extreme DeOxy (100% N₂) conditions.

The average delay time for all 6 patient samples tested were about 109 s±30 s during the initial DeOxy and decreased to 81 s±14 s during the fifth DeOxy cycle. This process was diffusion limiting, which was slower than the polymerization process of HbS in solution and in cells induced by pohotolysis of intracellular carboxy hemoglobin with an argon ion laser focused inside the cell.

Morphology analysis indicated a lack of “apparent plastic deformation” in cell membrane during the repetitive hypoxia. Evidence is that fully sickled RBCs always recovered to their initial relaxed state with cell membrane visually intact after each cycle of ReOxy. This may have been due to the limited hypoxia cycles in the present study (5 cycles in 30 minutes) compared to 2×10⁴ cycles in a typical 15-day lifespan of sickle cells in vivo. Another possible explanation lies in the “free suspension” condition in our in vitro assay, which is less severe than the in vivo circulation condition involved with additional complicated flow dynamics and cell-cell interactions in the vasculatures.

### Conclusions

The microfluidic assay described herein provided a cyclic hypoxia model that mimics the DeOxy and ReOxy process during in vivo circulation. This is in contrast to the existing approaches in mimicking the microenvironment for cell sickling studies. A microfluidic hypoxia assay, such as an assay provided herein, can be utilized as a novel accelerated damage model using cycles of hypoxia in the study of impacts of varied oxygen levels on cell sickling. Quantitative measurement of the kinetics of cell sickling in response to repetitive hypoxia conditions indicated a presence of “memory” in kinetics of cell sickling but an absence of “memory” in sickling shape. The duration of DeOxy and ReOxy periods can be adjusted to mimic oxygen changes in varied blood circulation speeds. These data provide a basis for studying the joint influences of shear stress and intracellular HbS polymerization on sickle cell pathology.

### REFERENCES

1. Ballas, S. K., Gupta, K., & Adams-Graves, P. (2012) Sickle cell pain: a critical reappraisal. Blood 120(18):3647-3656.
2. Castro, O., et al. (1994) The Acute Chest Syndrome in Sickle-Cell Disease - Incidence and Risk-Factors. Blood 84(2):643-649.
3. Van, E. J. B., Van Der, A. G., Tuijn Van, C. F. J., Schnog, J. J., & Biemond, B. J. (2006) Systematic evaluation of sickle cell-related organ damage and complications: Implications for sickle cell disease management. Blood 108(11):25b-25b.
4. Pegelow, C. H., et al. (1997) Natural history of blood pressure in sickle cell disease: Risks for stroke and death associated with relative hypertension in sickle cell anemia. Am J Med 102(2):171-177.
5. Machado, R. F., Hildescheim, M., Mendelsohn, L., Kato, G. J., & Gladwin, M. T. (2009) NT-Pro Brain Natriuretic Peptide Levels and the Risk of Stroke and Death in the Cooperative Study of Sickle Cell Disease. Blood 114(22):617-617.
6. Embury, S. H. (2004) The not-so-simple process of sickle cell vasoocclusion. Microcirculation 11 (2): 101-113.
7. Frenette, P. S. (2002) Sickle cell vaso-occlusion: multistep and multicellular paradigm. Curr Opin Hematol 9(2):101-106.
8. Barabino, G. A., Platt, M. O. & Kaul, D. K. (2010) Sickle Cell Biomechanics. Annu Rev Biomed Eng 12:345-367.
9. Eaton, W. A. & Hofrichter, J. (1987) Hemoglobin-S Gelation and Sickle-Cell Disease. Blood 70(5):1245-1266.
10. Mozzarelli, A., Hofrichter, J., & Eaton, W. A. (1987) Delay Time of Hemoglobin-S Polymerization Prevents Most Cells from Sickling Invivo. Science 237(4814):500-506.
11. Eaton, W. A., Hofrichter, J., & Ross, P. D. (1976) Delay Time of Gelation—Possible Determinant of Clinical Severity in Sickle-Cell Disease. Blood 47(4):621-627.
12. Higgins, J. M., Eddington, D. T., Bhatia, S. N., & Mahadevan, L. (2007) Sickle cell vasoocclusion and rescue in a microfluidic device. P Natl Acad Sci USA 104(51):20496-20500.
13. Dhananjaya K. Kaul, R. L. N., Silvio Baez (1983) Pressure Effects on the Flow Behavior of Sickle (HbSS) Red Cells in Isolated (ex-1/Go) Microvascular System 26:170-181.
14. Platt, O. S., et al. (1991) Pain in Sickle-Cell Disease—Rates and Risk-Factors. New Engl J Med 325(1):11-16.
15. Platt, O. S., et al. (1994) Mortality in Sickle-Cell Disease—Life Expectancy and Risk-Factors for Early Death. New Engl J Med 330(23):1639-1644.
16. Powars, D. R., Schroeder, W. A., Weiss, J. N., Chan, L. S., & Azen, S. P. (1980) Lack of Influence of Fetal Hemoglobin Levels or Erythrocyte Indexes on the Severity of Sickle-Cell-Anemia. J Clin Invest 65(3):732-740.
17. Poillon, W. N., Kim, B. C., & Castro, O. (1998) Intracellular hemoglobin S polymerization and the clinical severity of sickle cell anemia. Blood 91(5):1777-1783.

18. Billett, H. H., Kim, K., Fabry, M. E., & Nagel, R. L. (1986) The Percentage of Dense Red-Cells Does Not Predict Incidence of Sickle-Cell Painful Crisis. Blood 68(1):301-303.
19. Odenheimer, D. J., Sarnaik, S. A., Whitten, C. F., Rucknagel, D. L., & Sing, C. F. (1987) The Relationship between Fetal Hemoglobin and Disease Severity in Children with Sickle-Cell-Anemia. Am J Med Genet 27(3):525-535.
20. Akinsheye, I., et al. (2011) Fetal hemoglobin in sickle cell anemia. Blood 118(1):19-27.
21. Lande, W. M., et al. (1988) The Incidence of Painful Crisis in Homozygous Sickle-Cell Disease—Correlation with Red-Cell Deformability. Blood 72(6):2056-2059.
22. Ballas, S. K., et al. (1988) Rheologic Predictors of the Severity of the Painful Sickle-Cell Crisis. Blood 72(4):1216-1223.
23. Ballas, S. K. & Smith, E. D. (1992) Red-Blood-Cell Changes during the Evolution of the Sickle-Cell Painful Crisis. Blood 79(8):2154-2163.
24. Wood, D. K., Soriano, A., Mahadevan, L., Higgins, J. M., & Bhatia, S. N. (2012) A Biophysical Indicator of Vaso-occlusive Risk in Sickle Cell Disease. Sci Transl Med 4(123).
25. Abbyad, P., Tharaux, P. L., Martin, J. L., Baroud, C. N., & Alexandrou, A. (2010) Sickling of red blood cells through rapid oxygen exchange in microfluidic drops. Lab Chip 10(19):2505-2512.
26. Tsai, M., et al. (2012) In vitro modeling of the microvascular occlusion and thrombosis that occur in hematologic diseases using microfluidic technology. J Clin Invest 122(1):408-418.
27. Asakura, T., et al. (1994) Partially Oxygenated Sickled Cells—Sickle-Shaped Red-Cells Found in Circulating Blood of Patients with Sickle-Cell Disease. P Natl Acad Sci USA 91(26):12589-12593.
28. Itoh, T., Chien, S., & Usami, S. (1992) Deformability Measurements on Individual Sickle Cells Using a New System with Po2 and Temperature Control. Blood 79(8):2141-2147.
29. Takashima, S., Chang, S., & Asakura, T. (1985) Shape Change of Sickled Erythrocytes Induced by Pulsed Rf Electrical Fields. P Natl Acad Sci USA 82(20):6860-6864.
30. Kueh, H. Y., Brieher, W. M., & Mitchison, T. J. (2008) Dynamic stabilization of actin filaments. P Natl Acad Sci USA 105(43):16531-16536.
31. Christoph, G. W., Hofrichter, J., & Eaton, W. A. (2005) Understanding the shape of sickled red cells.Biophys J88(2):1371-1376.
32. Coletta, M., Hofrichter, J., Ferrone, F. A., & Eaton, W. A. (1982) Kinetics of Sickle Hemoglobin Polymerization in Single Red-Cells.Nature 300(5888):194-197.
33. Kniazeva, T., Hsiao, J. C., Charest, J. L., & Borenstein, J. T. (2011) A microfluidic respiratory assist device with high gas permeance for artificial lung applications. Biomed Microdevices 13(2):315-323.
34. Kaul, D. K. & Xue, H. (1991) Rate of Deoxygenation and Rheologic Behavior of Blood in Sickle-Cell-Anemia. Blood 77(6):1353-1361.
35. Abdulmalik, 0., et al. (2005) 5-hydroxymethyl-2-furfural modifies intracellular sickle haemoglobin and inhibits sickling of red blood cells. Brit J Haematol 128(4):552-561.
36. Itoh, T., Chien, S., & Usami, S. (1995) Effects of Hemoglobin Concentration on Deformability of Individual Sickle Cells after Deoxygenation. Blood 85(8):2245-2253.
37. Kaul, D. K. & Liu, X. D. (1999) Rate of deoxygenation modulates rheologic behavior of sickle red blood cells at a given mean corpuscular hemoglobin concentration. Clin Hemorheol Micro 21(2):125-135.
38. Corbett, J. D., Mickols, W. E., & Maestre, M. F. (1995) Effect of Hemoglobin Concentration on Nucleation and Polymer Formation in Sickle Red-Blood-Cells. J Biol Chem 270(6):2708-2715.
39. Schwartz, R. S., Musto, S., Fabry, M. E., & Nagel, R. L. (1998) Two distinct pathways mediate the formation of intermediate density cells and hyperdense cells from normal density sickle red blood cells. Blood 92(12):4844-4855.
40. Brugnara, C. (1995) Erythrocyte dehydration in pathophysiology and treatment of sickle cell disease. 2(2):132-8.
41. Makhijani, V. B. & Cokelet, G. R. (1994) In-Vivo Polymerization of Sickle-Cell Hemoglobin—a Theoretical-Study. Blood Cells 20(1):169-183.
42. Vekilov, P. G. (2007) Sickle-cell haemoglobin polymerization: is it the primary pathogenic event of sickle-cell anaemia? Brit J Haematol 139(2):173-184.
43. Basu, S., et al. (2011) F-cell levels are altered with erythrocyte density in sickle cell disease. Blood Cell Mol Dis 47(2):117-119.
44. Yasin, Z., et al. (2003) Phosphatidylsefine externalization in sickle red blood cells: associations with cell age, density, and hemoglobin F. Blood 102(1):365-370.
45. van Beers, E. J., et al. (2014) Imaging flow cytometry for automated detection of hypoxia-induced erythrocyte shape change in sickle cell disease. Am J Hematol 89(6):598-603.
46. Charache, S., et al. (1995) Effect of Hydroxyurea on the Frequency of Painful Crises in Sickle-Cell-Anemia. New Engl JMed 332(20):1317-1322.
47. Davies, S. C. & Gilmore, A. (2003) The role of hydroxyurea in the management of sickle cell disease. Blood Rev 17(2):99-109.
48. Rodgers, G. P., Dover, G. J., Noguchi, C. T., Schechter, A. N., & Nienhuis, A. W. (1990) Hematologic Responses of Patients with Sickle-Cell Disease to Treatment with Hydroxyurea. New Engl JMed 322(15):1037-1045.
49. Charache, S., et al. (1992) Hydroxyurea—Effects on Hemoglobin-F Production in Patients with Sickle-Cell-Anemia. Blood 79(10):2555-2565.
50. Rees, D. C., Williams, T. N., & Gladwin, M. T. (2010) Sickle-cell disease. Lancet 376(9757):2018-2031.
51. Steinberg, M. H., Chui, D. H. K., Dover, G. J., Sebastiani, P., & Alsultan, A. (2014) Fetal hemoglobin in sickle cell anemia: a glass half full? Blood 123(4):481-485.
52. Segel, G. B., Simon, W., & Lichtman, M. A. (2011) Should We Still Be Focused on Red Cell Hemoglobin F as the Principal Explanation for the Salutary Effect of Hydroxyurea in Sickle Cell Disease? Pediatr Blood Cancer 57(1):8-9.
53. Steinberg, M. H., et al. (1997) Fetal hemoglobin in sickle cell anemia: Determinants of response to hydroxyurea. Blood 89(3):1078-1088.
54. Fertrin, K. Y., et al. (2013) Imaging Flow Cytometry Documents Incomplete Resistance Of F-Cells To Hypoxia-Induced Sickling In Blood Samples From Patients With Sickle Cell Anemia. Blood 122(21).
55. Orringer, E. P., et al. (1991) Effects of Hydroxyurea on Hemoglobin-F and Water-Content in the Red-Blood-Cells of Dogs and of Patients with Sickle-Cell-Anemia. Blood 78(1):212-216.
56. Ballas, S. K., Dover, G. J., & Charache, S. (1989) Effect of Hydroxyurea on the Rheological Properties of Sickle Erythrocytes Invivo. Am J Hematol 32(2):104-111.
57. Uyuklu, M., Meiselman, H. J., & Baskurt, O. K. (2009) Effect of hemoglobin oxygenation level on red blood cell deformability and aggregation parameters. 41(3):179-188.
58. Kaul, D. K., Finnegan, E., & Barabino, G. A. (2009) Sickle Red Cell-Endothelium Interactions. Microcirculation 16(1):97-111.
59. Barabino, G. A., Mcintire, L. V., Eskin, S. G., Sears, D. A., & Udden, M. (1987) Endothelial-Cell Interactions with Sickle-Cell, Sickle Trait, Mechanically Injured, and Normal Erythrocytes under Controlled Flow. Blood 70(1):152-157.
60. Kaul, D. K., Fabry, M. E., & Nagel, R. L. (1989) Microvascular Sites and Characteristics of Sickle-Cell Adhesion to Vascular Endothelium in Shear-Flow Conditions—Pathophysiological Implications. P Natl Acad Sci USA 86(9):3356-3360.
61. Kaul, D. K. & Fabry, M. E. (2004) In vivo studies of sickle red blood cells. Microcirculation 11(2):153-165.
62. Lei, H. & Karniadakis, G. E. (2013) Probing vasoocclusion phenomena in sickle cell anemia via mesoscopic simulations. 110(28):11326-11330.
63. Constance Tom Noguchi, Dennis A. Torchia, & Schechter AN (1983) Intracellular Polymerization of Sickle Hemoglobin: Effects of Cell heterogeneity. The Journal of Clinical Investigation 72:846-852.
64. Abdulmalik O, et al. (2005) 5-hydroxymethyl-2-furfural modifies intracellular sickle haemoglobin and inhibits sickling of red blood cells. Brit J Haematol 128(4):552-561.
65. Stern W, et al. (2013) Ase 1, First-in-Man, Dose-Response Study of Aes-103 (5-Hmf), an Anti-Sickling, Allosteric Modifier of Hemoglobin Oxygen Affinity in Healthy Normal Volunteers. Am J Hematol 88(12):E79-E79.
66. Mendelsohn LG, et al. (2012) Effect of Aes-103 Anti-Sickling Agent On Oxygen Affinity and Stability of Red Blood Cells From Patients with Sickle Cell Anemia. Blood 120(21).

### OTHER EMBODIMENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one or all of the group members are present in, employed in or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A high throughput method of measuring a property of an individual cell under controlled gas conditions, comprising:
flowing a fluid comprising a plurality of cells through one or more constrictions;
obtaining a measurement of an individual cell in the fluid; and
regulating a level of gas in the fluid.

2. (canceled)

3. The method of claim 1, wherein the property is deformability, rigidity, viscoelasticity, viscosity or adhesiveness.

4-14. (canceled)

15. The method of claim 1, wherein the property is sickling, sphericity change, aspect ratio change, or change in cell texture.

16-24. (canceled)

25. The method of claim 1, wherein the cells comprise red blood cells, white blood cells, stem cells or epithelial cells.

26-29. (canceled)

30. The method of claim 1, wherein the level of the gas in the fluid is regulated to be at a concentration from 5% to 20%.

31-43. (canceled)

44. The method of claim 1, wherein the cells are from a subject having or suspected of having a condition or disease selected from the group consisting of sickle cell disease (SCD), sickle cell trait (SCT), spherocytosis, ovalocytosis, alpha thalassemia, beta thalassemia, delta thalassemia, malaria, anemia, diabetes and leukemia.

45-49. (canceled)

50. The method of claim 1, wherein the property is measured after one or more reoxygenation (ReOxy) cycles.

51. (canceled)

52. The method of claim 1, wherein the property is measured after one or more deoxygenation (DeOxy) cycles.

53-60. (canceled)

61. A microfluidic device comprising:
(a) a structure defining one or more microfluidic channels that each comprise
(i) a first constriction having a first inlet orifice and a first outlet orifice, wherein the first inlet orifice is geometrically different from the first outlet orifice; and
(b) a wall adjacent to the microfluidic channel, wherein at least a portion of the wall comprises a gas permeable membrane or film.

62. The device of claim 61, wherein the one or more microfluidic channels each also comprise (ii) a second constriction having a second inlet orifice and a second outlet orifice.

63-64. (canceled)

65. The device of claim 62, wherein the first constriction is arranged in series with the second constriction such that a flow path through the first constriction is longitudinally aligned with a flow path through the second constriction.

66. (canceled)

67. The device of claim 65, wherein the one or more microfluidic channels further comprise a gap region between the first constriction and the second constriction.

68-106. (canceled)

107. The device of claim 61, further comprising:
a reservoir fluidically connected with the one or more microfluidic channels, and
a pump that perfuses fluid from the reservoir through the one or more microfluidic channels.

108-118. (canceled)

119. The device of claim 61, further comprising a gas channel, wherein the gas channel contacts the gas permeable membrane or film.

120. (canceled)

121. The device of claim 119, wherein the gas channel comprises an inlet and/or an outlet.

122. (canceled)

123. The device of claim 61, wherein the gas permeable membrane or film is made of polydimethylsiloxane (PDMS), hydroxypropyl cellulose (HPC), hydroxypropyl methylcellulose (HPMC), cellulose triacetate (CTA), or poly(methyl methacrylate) (PMMA).

124. (canceled)

125. A method for analyzing a condition or disease in a subject, the method comprising:
(a) perfusing a fluid comprising one or more cells from the subject through the device of claim 61;
(b) determining a property of one or more of the cells; and
(c) comparing the property to an appropriate standard, wherein the results of the comparison are indicative of the status of the condition or disease in the subject.

126-139. (canceled)

140. A method for monitoring the effectiveness of a therapeutic agent for treating a disease or condition in a subject comprising:
(a) perfusing a fluid comprising one or more cells from the subject through the device of claim 61;
(b) determining a property of one or more of the cells;
(c) treating the subject with the therapeutic agent; and
(d) repeating steps (a) and (b) at least once wherein a difference in the property of one or more cells is indicative of the effectiveness of the therapeutic agent.

141. A method for determining the effectiveness of a therapeutic comprising:
(a) obtaining a biological sample from a subject comprising a cell;
(b) perfusing a fluid comprising one or more cells from the subject through the device of claim 61;
(c) determining a property of one or more of the cells;
(d) contacting the biological sample comprising a cell with the therapeutic;
(e) perfusing a fluid comprising the product of (d) through the device of claim 61;
(f) determining a property of one or more of the cells from (e); and
(g) comparing the property of one or more cells from (c) with the property of one or more cells from (f), wherein the results of the comparison are indicative of the effectiveness of the therapeutic.

142-143. (canceled)

144. A real-time method for quantifying cell morphological kinetics in response to varying levels of gas comprising:
(a) perfusing a fluid comprising one or more blood cells through the device of claim 61, wherein the fluid has a first level of gas;
(b) determining a property of one or more of the cells from (a);
(c) perfusing a fluid comprising one or more cells through the device of claim 61; wherein the fluid has a second level of gas that is different from the first level;
(d) determining a property of one or more of the cells from (c); and
(e) quantifying the cell morphological kinetics of the cells from (b) and (d).

145. (canceled)
